(19) United States
(12) Patent Application Publication
(10) Pub. No.: US 2025/0177359 A1
(43) Pub. Date: Jun. 5, 2025

(54) COMPOUNDS FOR USE IN THE TREATMENT OF CANCER AND INFLAMMATORY CONDITIONS

(71) Applicants: Oulun Yliopisto, Oulun Yliopisto (FI); Università degli Studi di Perugia, Perugia (IT)

(72) Inventors: Lari Lehtiö, Oulu (FI); Mirko Maksimainen, Oulu (FI); Sudarshan Narasimha Murthy, Oulu (FI); Oriana Tabarrini, Perugia (IT); Maria Giulia Nizi, Perugia (IT); Serena Massari, Perugia (IT)

(21) Appl. No.: 18/834,003

(22) PCT Filed: Jan. 30, 2023
(86) PCT No.: PCT/FI2023/050060

§ 371 Date: Jul. 29, 2024

(30) Foreign Application Priority Data
Jan. 28, 2022 (FI) 20225073

(51) Int. Cl. A61K 31/429; A61K 31/437; A61P 35/00

(52) U.S. Cl. CPC A61K 31/429; A61K 31/437; A61P 35/00

(57) ABSTRACT

According to an aspect of the present invention, there is provided a [1,2,4]triazolo[3,4-b]benzothiazole scaffold, which can be used to inhibit efficiently human PARP enzymes. The compounds disclosed bind to the nicotinamide pocket of the enzyme and compete with the natural substrate, NAD*. The present compounds are new types of nicotinamide mimics with wide use as new therapeutics especially against cancer.

### FIELD

The present invention relates to the field of enzyme inhibitors useful for cancer treatments.

### BACKGROUND

ADP-ribosylation is a post-translational modification found in bacteria and eukaryotes. Enzymes of the human diphtheria toxin-like ARTD enzyme family, also called PARPs, can catalyze both mono-ADP-ribosylation (MAR, mono-ARTs) as well as generate elongated and branched chains of poly-ADP-ribose (PAR, poly-ARTs). The PARP and tankyrase (TNKS) enzymes form the ARTD family of structurally and functionally diverse enzymes, which are involved in the regulation of various key biological and pathological processes such as DNA repair, cell differentiation, gene transcription, signal transduction pathways.

PARPs and TNKSs use nicotinamide adenine dinucleotide, NAD⁺, to transfer an ADP-ribose (ADPr) unit onto target proteins or nucleic acids with a release of nicotinamide. The transfer of ADPr in proteins occurs onto amino acid side chains with a nucleophilic oxygen, nitrogen, or sulfur resulting in N—, O—, or S-glycosidic linkage to the ADP-ribose. This can be further extended to poly-ADP-ribose by poly-ARTs PARP1-2 and TNKS1-2. Poly-ARTs contain a triad of amino acids H—Y-E in their active sites. H—Y being important in binding the NAD⁺, while E stabilizes the oxacarbenium ion transition state and enables the elongation of ADP-ribose chain by activating the ribose 2′-hydroxyl group. However, the H—Y-E motif is not an absolute indicator determining the PARylation activity as there are two enzymes, PARP3 and PARP4 having the H—Y-E motif but catalyzing a transfer of a single ADPr unit (MAR).

MARylation catalyzed by mono-ARTs is associated with various cellular processes. Mono-ARTs have been shown to, for example, regulate neuronal growth, response to environmental toxins, affect stem cells, immune responses and development of human diseases like cancer.

WO2017174879 discloses selective PARP10 inhibitors.

Growing interest in the development of PARP1 inhibitors has led to at least four marketed drugs for the treatment of advanced-stage cancers, such as PARP1-4 inhibitor Olaparib (Lynparza) for the treatment of ovarian cancer. Recent developments in the discovery and protection of novel PARP/TNKS inhibitors as anticancer agents are reviewed by Velagapudi et al., 2021 and Mehta & Bhatt, 2021.

### SUMMARY OF THE INVENTION

The present inventors have developed a [1,2,4]triazolo[3,4-b]benzothiazole scaffold, which can be used to inhibit efficiently human PARP enzymes. The compounds disclosed bind to the nicotinamide pocket of the enzyme and compete with the natural substrate, NAD⁺. The present compounds are new types of nicotinamide mimics with wide use as new therapeutics especially against cancer. During the structure-guided development of the structure activity relationship, a range of analogs was synthesized and it was shown that the compounds could inhibit with nM level potencies not only the mono-ARTs but also tankyrases and DNA activated PARPs. Tankyrases are drug targets due to their multiple roles in the cell but exemplified by the Wnt/β-catenin signaling often misregulated in cancers. Targeting DNA dependent PARPs allows specific killing of mutated cancer cells through synthetic lethality.

According to a first aspect of the present invention, there is provided a compound having a general formula

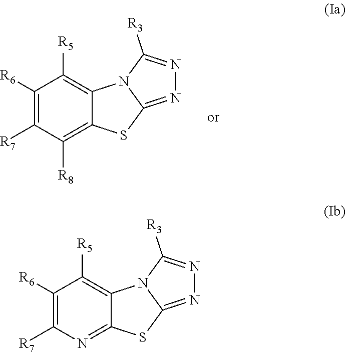

wherein:
R3, R5, R6, R7 and R8 are independently selected from the group consisting of: H, —NH₂, —COOH, —CN, —NO₂, —SH, —OH, F, Cl, Br, I, C₁₋₄ alkyl, aryl, C₁₋₄ alkyl aryl, C₁₋₄ alkyl halide, C₁₋₄ amino alkyl, aryl halide, C₁₋₄ alkyl aryl halide, alkoxy —OR, ester —OC(═O)R, amine —NHR, amide —NHC(═O)R, imine —(N=)R, NHSO₂(C₁₋₄ alkyl), and thioether —SR; wherein R is selected from the group consisting of: C₁₋₄ alkyl, aryl, C₁₋₄ alkyl aryl, C₁₋₄ alkylhalide, aryl halide, C₁₋₄ amino alkyl, C₃₋₆ cycloalkyl and C₁₋₄ alkyl aryl halide; or a pharmaceutically acceptable salt thereof, for use as a medicine.

In another aspect, the invention provides a human PARP/TNKS inhibitor as defined in the invention for use in the treatment of cancer.

In another aspect, the invention provides a human PARP/TNKS inhibitor as defined in the invention for use in the treatment of inflammatory disorders.

In another aspect, the invention provides a pharmaceutical composition comprising a human PARP/TNKS inhibitor as defined in the invention and at least a pharmaceutically acceptable buffer, carrier, excipient, preservative or stabilizer.

In another aspect, the invention provides a use of a human PARP/TNKS inhibitor as defined in the invention for the manufacture of a medicament.

In another aspect, the invention provides a method of treating cancer comprising a step of administering a human PARP/TNKS inhibitor as defined in the invention to a patient suffering from a cancer.

In another aspect, the invention provides a method of treating inflammatory disorder comprising a step of administering a human PARP/TNKS inhibitor as defined in the invention to a patient suffering from an inflammatory disorder.

In another aspect, the invention provides a use of a human PARP/TNKS inhibitor as defined in the invention as an ADP-ribosyltransferase inhibitor in vitro.

In another aspect, the invention provides an in vitro method for the inhibition of ADP-ribosyltransferase comprising a step of contacting a human PARP/TNKS inhibitor as defined in the invention with a sample suspected or known to comprise PARPs or TNKSs.

In another aspect, the invention provides an in vitro screening method for identifying inhibitors of PARP/TNKS comprising a step of contacting a human PARP/TNKS inhibitor as defined in the invention with ADP-ribosyltransferase, measuring the level of ADP-ribosyltransferase activity and selecting compounds with inhibit PARPs or TNKSs.

In another aspect, the invention provides a compound having a general formula

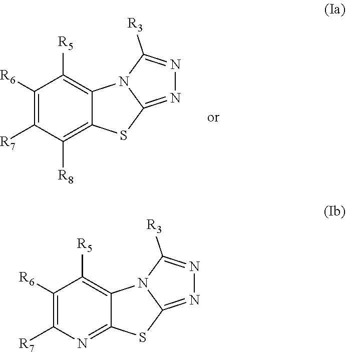

wherein:
R3, R5, R6, R7 and R8 are independently selected from the group consisting of: H, —NH₂, —COOH, —CN, —NO₂, —SH, —OH, F, Cl, Br, I, C₁₋₄ alkyl, aryl, C₁₋₄ alkyl aryl, C₁₋₄ alkyl halide, C₁₋₄ amino alkyl, aryl halide, C₁₋₄ alkyl aryl halide, alkoxy —OR, ester —OC(═O)R, amine —NHR, amide —NHC(═O)R, imine —(N=)R, NHSO₂(C₁₋₄ alkyl), and thioether —SR;
wherein R is selected from the group consisting of: C₁₋₄ alkyl, aryl, C₁₋₄ alkyl aryl, C₁₋₄ alkylhalide, aryl halide, C₁₋₄ amino alkyl, C₃₋₆ cycloalkyl and C₁₋₄ alkyl aryl halide; or a salt thereof,
wherein the following combinations of R3-R8 substitutions are excluded:
   R3 is H, R5 is H, R6 is H, R7 is H, and R8 is H;
   R3 is H, R5 is H, R6 is H, R7 is —CH₃, and R8 is H;
   R3 is H, R5 is H, R6 is H, R7 is Cl, and R8 is H;
   R3 is H, R5 is H, R6 is H, R7 is —OH, and R8 is H;
   R3 is H, R5 is H, R6 is H, R7 is —OCH₃, and R8 is H;
   R3 is H, R5 is —CH₃, R6 is H, R7 is —CH₃, and R8 is H;
   R3 is —OH, R5 is H, R6 is H, R7 is —CH₃, and R8 is H; and
   R3 is —SH, R5 is H, R6 is H, R7 is —CH₃, and R8 is H.
      More specifically, the invention is mainly characterized by what is stated in the independent claims.

### BRIEF DESCRIPTION OF THE DRAWINGS

FIG. 1. We have now investigated the shown OUL40 scaffold (i.e. [1,2,4]triazolo[3,4-b]benzothiazole scaffold) of Formula I by analog design and potency profiling against PARP and TNKS enzymes. We have also used protein crystallography to determine binding modes of the analogs and have found several compounds with very good potencies as PARP inhibitors.

FIG. 2. Analog OUL232 (Formula II) inhibits PARP15 (IC₅₀=56 nM) by binding to its nicotinamide pocket. OUL232 also inhibits efficiently PARP10 (IC₅₀=7.8 nM). We have also identified another analog, OUL243 (Formula III), which inhibits PARP10 (IC₅₀=25 nM) with a better potency compared to the PARP10 inhibitor, OUL35 (IC₅₀=329 nM, Venkannagari et al. 2016). In addition to the mono-ART members of the PARP family (like PARP10 and PARP15), the OUL40 scaffold is also promising to develop potent inhibitors for DNA-dependent PARPs as well as tankyrases (TNKSs). For instance, the OUL245 (Formula IV) analog inhibits PARP2 (IC₅₀=44 nM) and TNKS2 (IC₅₀=370 nM).

FIG. 3. (A) Potential human PARP inhibitor compounds were synthesized as shown in the scheme. Reagents and conditions: i) hydrazine hydrate, EtOH, 80° C., overnight; ii) NH₄SCN, H₂O, 12 N HCl, reflux, 6-48 h; iii) Br₂, CHCl₃, r.t., 2-8 h; iv) hydrazine hydrate, CH₃COOH, ethylene glycol, 125° C., 7-48 h; v) potassium ethyl xanthogenate, dry DMF, 110° C.; 3 h; vi) formic acid, reflux, 7-48 h; vii) BBr₃, dry CH₂Cl₂, r.t., 3 h; viii) CS₂, KOH, EtOH, reflux, 2 h; ix) Mel, K₂CO₃, dry DMF, 80° C., 2 h; x) p-Cl-benzyl chloride, EtOH, reflux, 4 h; xi) urea, neat, fusion, 3 h; xii) CNBr, EtOH, reflux, 3 h; xiii) p-Cl-benzaldehyde, p-TsOH, dry benzene, reflux, 16 h; xiv) acyl chloride, Et₃N, dry DMF, 80° C., 2-12 h; xv) NaBH₄, absolute EtOH, r.t., overnight; xvi) isobutyl alcohol, NaH, dry DMF, 80° C., 4 h; xvii) NH₄Cl, Zn, H₂O/acetone, 60° C., 3 h. (B) Listing of the Ar-structures in (A).

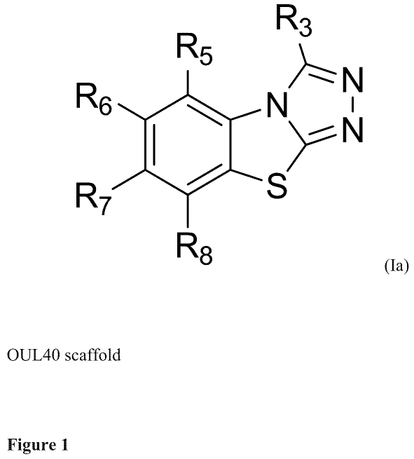

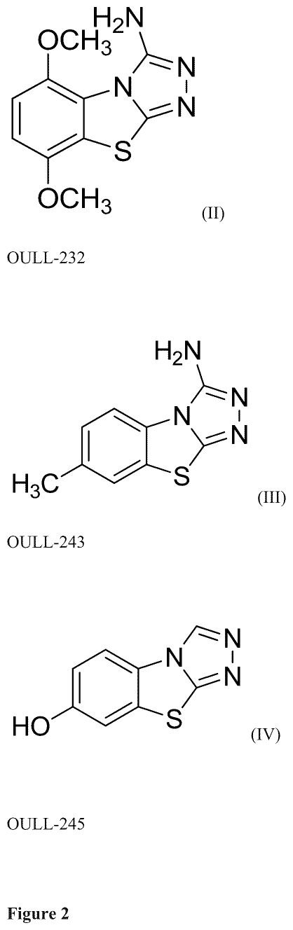

### EMBODIMENTS

ADP-ribosylation of proteins by human diphtheria toxin-like ADP-ribosyltransferases (PARPs/TNKSs) plays important roles in various activities ranging from cellular signaling, DNA repair and cell proliferation to the immune response. Here we describe small molecule inhibitors based on a [1,2,4]triazolo[3,4-b]benzothiazole scaffold.

The present invention is thus directed to a compound having a general formula

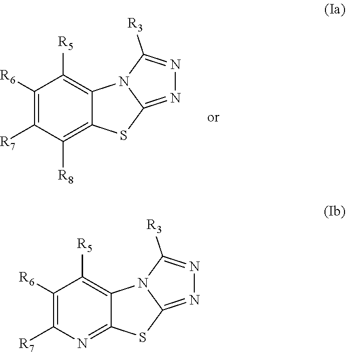

wherein:
R3, R5, R6, R7 and R8 are independently selected from the group consisting of: H, —NH₂, —COOH, —CN, —NO₂, —SH, —OH, F, Cl, Br, I, C₁₋₄ alkyl, aryl, C₁₋₄ alkyl aryl, C₁₋₄ alkyl halide, C₁₋₄ amino alkyl, aryl halide, C₁₋₄ alkyl aryl halide, alkoxy —OR, ester —OC(═O)R, amine —NHR, amide —NHC(═O)R, imine —(N=)R, NHSO₂(C₁₋₄ alkyl), and thioether —SR;
wherein R is selected from the group consisting of: C₁₋₄ alkyl, aryl, C₁₋₄ alkyl aryl, C₁₋₄ alkylhalide, aryl halide, C₁₋₄ amino alkyl, C₃₋₆ cycloalkyl and C₁₋₄ alkyl aryl halide;
or a salt thereof;
preferably for use as a medicine.

Preferably, R3 is selected from the group consisting of: H, —NH₂, —SH, —OH, F, Cl, Br, I, C₁₋₄ alkyl, aryl, C₁₋₄ alkyl aryl, C₁₋₄ alkyl halide, aryl halide, C₁₋₄ alkyl aryl halide, C₁₋₄ alkoxy halide, aryloxy halide, alkoxy —OR, ester OC(═O)R, amine —NHR, amide —NHCOR, imine —(N=)R, and thioether —SR;
wherein R is selected from the group consisting of: C₁₋₄ alkyl, aryl, C₁₋₄ alkyl aryl, C₁₋₄ alkylhalide, aryl halide, and C₁₋₄ alkyl aryl halide.

In other preferred embodiments, R3 of said compound is selected from the group consisting of H, NH₂, SH, OH, amine —NHR, amide —NHCOR, imine —(N=)R, and thioether —SR, wherein R is selected from the group consisting of: C₁₋₄ alkyl and C₁₋₄ alkyl aryl halide; R5, R6, R7 and R8 are independently selected from the group consisting of: H, F, Cl, Br, I, C₁₋₄ alkyl, C₁₋₄ alkoxy, C₁₋₄ alkyl halide, C₁₋₄ alkoxy halide, and OH

In preferred embodiments, the R5 of the said compound is selected from the group consisting of H, C₁₋₄ alkyl, C₁₋₄ alkoxy and F, OH; R6 is selected from the group consisting of H, C₁₋₄ alkyl and C₁₋₄ alkoxy; R7 is selected from the group consisting of H, OH, C₁₋₄ alkyl, and C₁₋₄ alkoxy and Cl; and R8 is selected from the group consisting of H and C₁₋₄ alkyl and C₁₋₄ alkoxy, OH. Preferably, the said C₁₋₄ alkyl is methyl, ethyl or isopropyl and said C₁₋₄ alkoxy is methoxy or ethoxy. More preferably, at least two of the groups R5-R8 are H.

In preferred embodiments, the imine in the said compound is

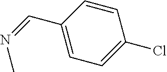

In preferred embodiments, the amine in the said compound is

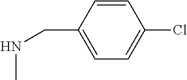

In preferred embodiments, the amide in the said compound is

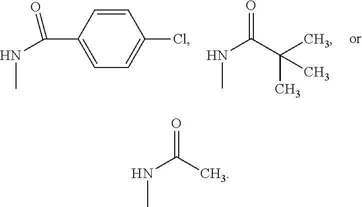

In preferred embodiments, the thioether in the said compound is selected from the group consisting of

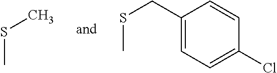

In other preferred embodiments, the R3 group of the said compound is amino group and the R5 and R8 groups are methoxy groups. More preferably, the said compound has the following formula

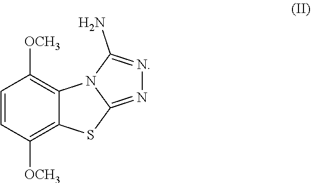 [SMILES: COc1ccc(OC)c2c1sc1nnc(N)n12]

In other preferred embodiments, the R3 group of the said compound is an amino group and the R7 group is a methyl group. More preferably, the said compound has the following formula

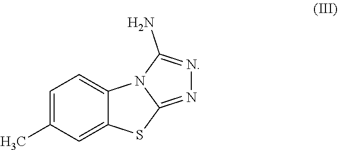

In other preferred embodiments, the R7 group of the said compound is a hydroxyl group. More preferably, the said compound has the following formula

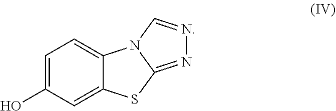 [SMILES: Oc1ccc2c(c1)sc1nncn12]

In other embodiments, the said compound preferably has following formula

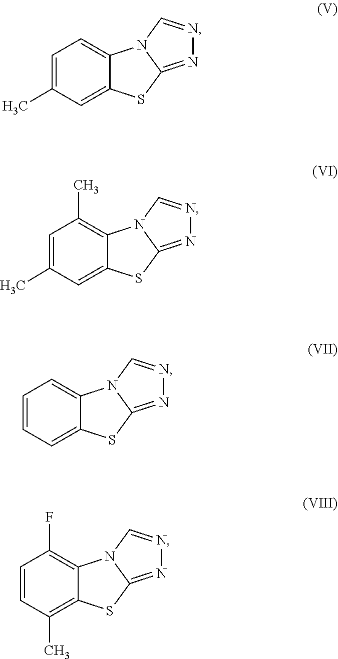

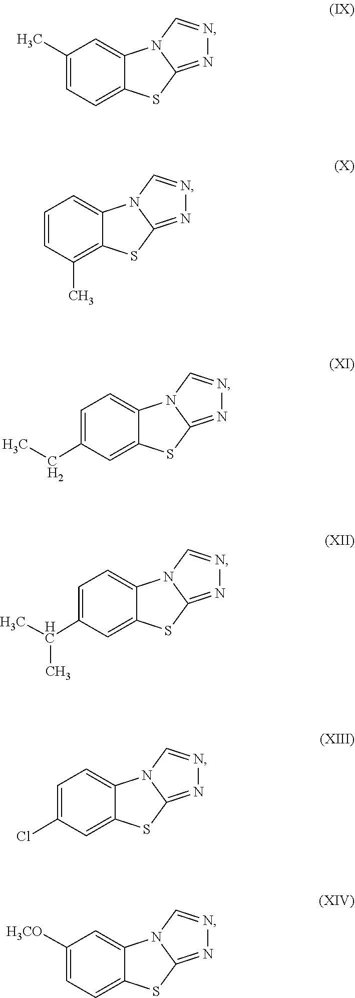

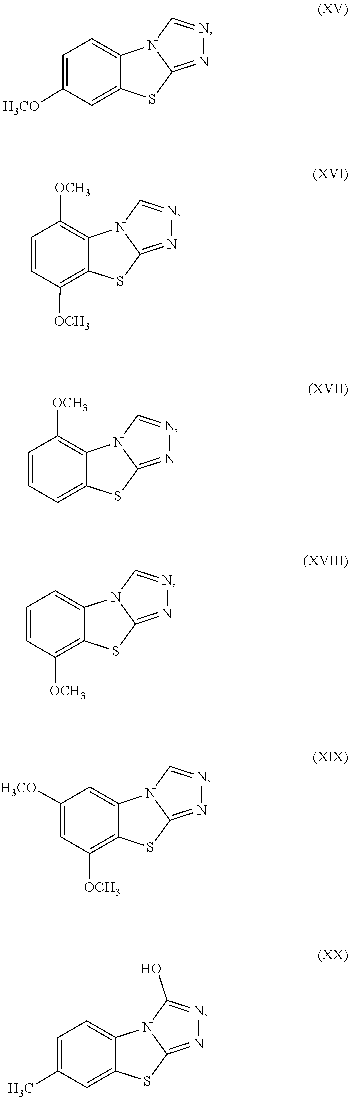 [SMILES: COc1ccc2c(c1)sc1nncn12 ; COc1ccc(OC)c2c1sc1nncn12 ; COc1cccc2sc3nncn3c12 ; COc1cccc2c1sc1nncn12 ; COc1cc(OC)c2sc3nncn3c2c1 ; Cc1ccc2c(c1)sc1nnc(O)n12]

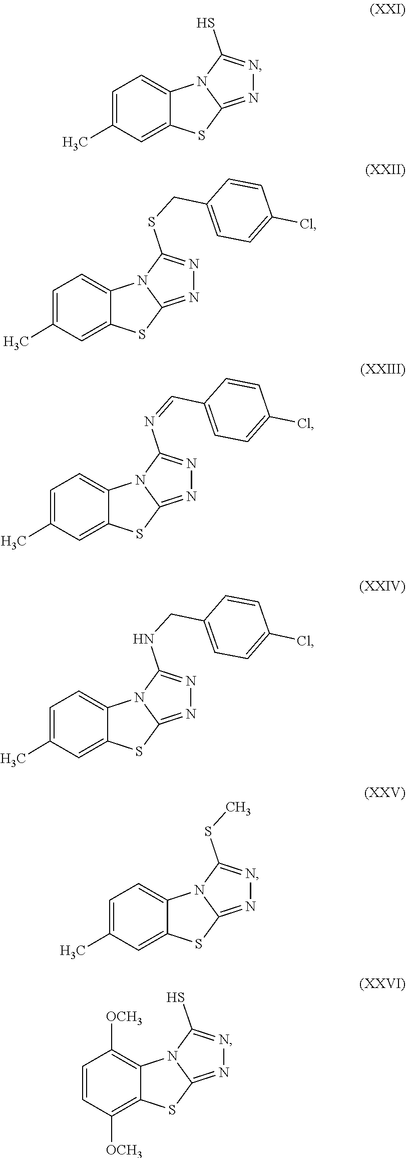 [SMILES: Cc1ccc2c(c1)sc1nnc(S)n12 ; Cc1ccc2c(c1)sc1nnc(SCc3ccc(Cl)cc3)n12 ; Cc1ccc2c(c1)sc1nnc(/N=C\c3ccc(Cl)cc3)n12 ; Cc1ccc2c(c1)sc1nnc(NCc3ccc(Cl)cc3)n12 ; CSc1nnc2sc3cc(C)ccc3n12 ; COc1ccc(OC)c2c1sc1nnc(S)n12]

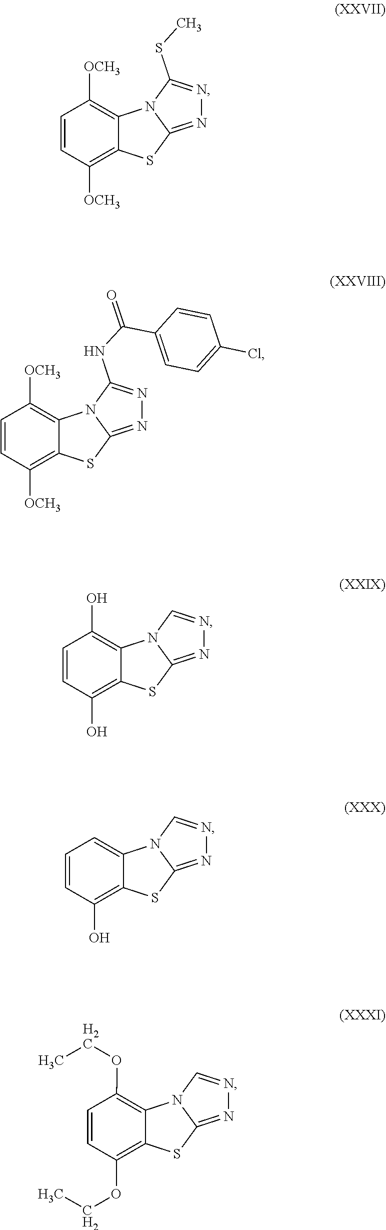 [SMILES: COc1ccc(OC)c2c1sc1nnc(SC)n12 ; COc1ccc(OC)c2c1sc1nnc(NC(=O)c3ccc(Cl)cc3)n12 ; Oc1ccc(O)c2c1sc1nncn12 ; Oc1cccc2c1sc1nncn12 ; CCOc1ccc(OCC)c2c1sc1nncn12]

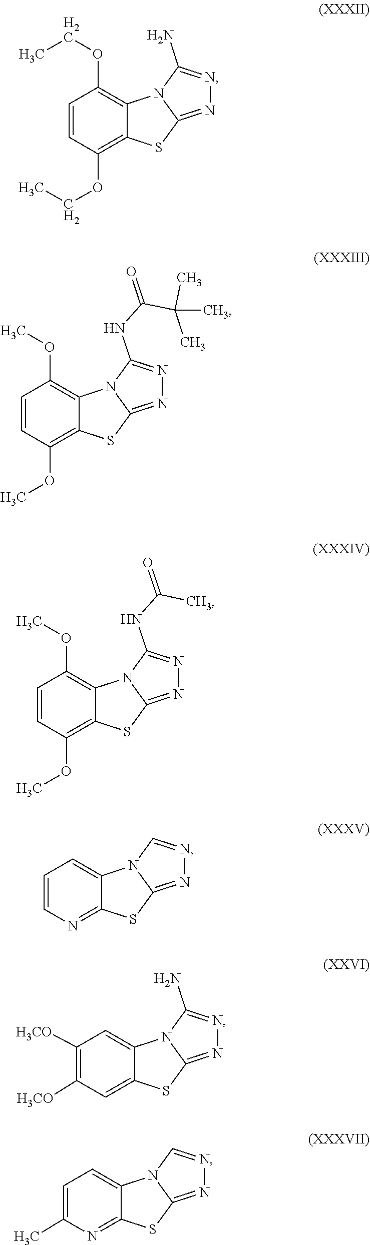 [SMILES: CCOc1ccc(OCC)c2c1sc1nnc(N)n12 ; COc1ccc(OC)c2c1sc1nnc(NC(=O)C(C)(C)C)n12 ; COc1ccc(OC)c2c1sc1nnc(NC(C)=O)n12 ; c1cnc2sc3nncn3c2c1 ; COc1cc2sc3nnc(N)n3c2cc1OC ; Cc1ccc2c(n1)sc1nncn12]

As used herein, “alkyl” is intended to include both branched and straight-chain aliphatic hydrocarbon groups having the specified number of carbon atoms. For example, “C₁₋₄ alkyl” is defined to include groups having 1, 2, 3 or 4 carbon atoms in a linear or branched arrangement. Alkyl groups may include heteroatoms, for example nitrogen, oxygen, sulfur and substituents such as halogens. Preferred alkyl groups are methyl, ethyl and iso-propyl groups.

As used herein, “aryl” refers to aromatic structures with 5 to 10 hydrocarbons. An aryl group can be unsubstituted or substituted. The ring atoms may include one or more heteroatoms, including but not limited to oxygen, nitrogen, sulfur, and selenium. Examples of aryl groups include, but are not limited to, phenyl, tolyl, xylyl, pyridyl, naphthyl, indanyl, thiazolyl, and furanyl.

The term “alkoxy” represents an alkyl group of indicated number of carbon atoms attached to the remainder of the molecule through an oxygen bridge. An example of suitable alkoxy groups is C₁₋₃ alkoxy, which includes methoxy, ethoxy and propoxy. Likewise, “aryloxy” refers to an aryl group of indicated number of carbon atoms attached to the remainder of the molecule through an oxygen bridge. Alkoxy groups may include heteroatoms, for example nitrogen, oxygen, sulfur and substituents such as halogens.

As used herein, “alkyl aryl” refers to a residue in which an aryl moiety is attached to the parent structure via an alkyl residue. Examples include but are not limited to benzyl, phenethyl, phenyl vinyl and phenyl allyl.

As used herein, “halogen-substituted” refers to structures where one or more hydrogen atoms have been replaced by halogen atoms. The term “halogen” or “halide” refers to fluorine, chlorine, bromine and iodine. For example, the term “alkyl halide” is defined to include groups having a stable straight or branched chain hydrocarbon consisting of at least one carbon atom and at least one halogen connected to the hydrocarbon chain.

As used herein, “thioether” is intendent to include structures where a sulfur atom is attached to an alkyl or aryl group. These alkyl and aryl groups may include heteroatoms, for example nitrogen, oxygen, sulfur and substituents such as halogens. Examples include but are not limited to methyl sulfide, ethyl sulfide, phenyl sulfide and p-chlorobenzyl sulfide.

As used herein, “amine” is intendent to include structures where a nitrogen atom is attached to an alkyl or aryl group. These alkyl and aryl groups may include one or more heteroatoms, including but not limited to oxygen, nitrogen, sulfur, and selenium. Examples include but are not limited to 4-chlorobenzamine. Examples also include tautomers of amines such as imines.

As used herein, “amide” is intendent to include structures where an amide group is attached to an alkyl or aryl group. These alkyl and aryl groups may include one or more heteroatoms, including but not limited to oxygen, nitrogen, sulfur, and selenium. Examples include but are not limited to 4-chlorobenzamide. Examples also include tautomers of amides such as imides.

As used herein, “imide” is intendent to include structures where an imide group is attached to an alkyl or aryl group. These alkyl and aryl groups may include one or more heteroatoms, including but not limited to oxygen, nitrogen, sulfur, and selenium. Examples include but are not limited to 4-chlorobenzimide (4-chloro benzencarboximidic acid).

The compounds as defined in Formulas Ia and Ib may have asymmetric or chiral structures and thus occur as racemates, racemic mixtures, or as individual diastereomers, or with all possible isomers and mixtures thereof, including optical isomers, all such stereoisomers being in the scope of this description. In addition, the compounds disclosed herein may exist as tautomers and both tautomeric forms are intended to be encompassed by the scope of this description, even though only one tautomeric structure is depicted. For instance, amine forms of R-groups may be specifically listed but tautomeric imine forms are also encompassed.

Other forms included in the above are the well-known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—NH₃⁺), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Preferably, the present compounds are for use in the treatment of cancer. Preferred applications would be in the treatment of breast cancer, ovarian cancer, prostate cancer, colorectal cancer, melanoma and leukemia.

Other cancers treatable by the present compounds are carcinomas such as bladder, kidney, liver, pancreas, lung, including small cell lung cancer, esophagus, gall-bladder, stomach, cervix, thyroid, and skin carcinoma, including squamous cell carcinoma; tumors of mesenchymal origin, including fibrosarcoma and rhabdomyosarcoma; tumors of the central and peripheral nervous system, including astrocytoma neuroblastoma, glioma and schwannomas; other tumors, including seminoma, teratocarcinoma, xeroderma pigmentosum, keratoxanthoma, thyroid follicular cancer and Kaposi's sarcoma.

In preferred embodiments of the invention, the present compounds are used together with a chemotherapeutic agent such as a DNA damaging compound and/or with radiotherapy. Preferred DNA-damaging anticancer compounds are platinum-based compounds, such as cisplatin, carboplatin, oxaliplatin, and picoplatin, and anthracyclines such as doxorubicin and daunorubicin and also methotrexate. Other preferred DNA-damaging anticancer compounds are topoisomerase I inhibitors such as irinotecan, topotecan, camptothecin and lamellarin D.

Accordingly, the present compounds are useful in combination with anti-cancer agents, such as checkpoint inhibitors or chemotherapeutic agents. The compounds of Formulas Ia and Ib may be useful as chemo- and radio-sensitizers for cancer treatment. They are useful for the treatment of patients who have previously undergone or are presently undergoing treatment for cancer. Such previous treatments include prior chemotherapy, radiotherapy, surgery or immunotherapy.

PARP inhibitors have been demonstrated as being useful for treatment of inflammatory disorders, therefore the present compounds are also for use in the treatment of inflammatory disorders such as acne vulgaris, asthma, autoimmune diseases, autoinflammatory diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

In an embodiment, the present invention also relates to pharmaceutical compositions which contain an inhibitor of the Formula I-XLII or a pharmaceutically acceptable salt thereof as active ingredient. These pharmaceutical compositions are for example those for enteral, such as in particular oral, those for parenteral administration, and those for local administration to a patient

The pharmaceutical compositions according to the invention usually contain the pharmacologically active ingredient according to Formula I-XLII together with known pharmaceutically acceptable buffers, carriers, excipients, diluents, adjuvants, fillers, buffers, stabilisers, preservatives or lubricants. The amount of the active ingredient in the pharmaceutical compositions according to the invention is, for example, from about 0.001% to 100% by weight, preferably from about 0.1% to about 50% by weight. The dose of the active ingredient can depend on various factors, such as the efficacy of the active ingredient, severity of the disease to be treated or its symptoms, administration procedure, sex, age, weight and/or individual condition of the subject in need of the treatment. In a normal case, for a human adult of about 75 kg in weight, one daily dose of about 1 mg to about 1000 mg, in particular from about 10 mg to about 500 mg, is to be estimated. This can be administered as a single dose or in several sub-doses.

In an embodiment, the pharmaceutical compositions may be in the form of an injectable aqueous solution. The injectable preparation may also be an injectable oil-in-water microemulsion where the active ingredient is dissolved in the oily phase. The injectable solutions or microemulsions may be introduced into a patient's blood stream by local bolus injection. The pharmaceutical compositions may be in the form of a sterile injectable aqueous or oleagenous suspension for intramuscular and subcutaneous administration. This suspension may be formulated according to the known art using suitable dispersing or wetting agents and suspending agents.

The preparation of typical pharmaceutically acceptable salts is described by Berge et al. (1977) J. Pharm. Sci. “Pharmaceutical Salts”, 66: 1-19.

The pharmaceutical composition described herein are intended for use in the treatment of cancer or inflammatory disorders.

In an embodiment, the present invention is also directed to the use of the compound as defined by Formula I-XLII for the manufacture of a medicament, preferably for the treatment of cancer or inflammatory disorders. Advantageously, said compound is used together with a chemotherapeutic agent and/or with radiotherapy as described above.

In an embodiment, the present invention is directed to a method of treating cancer or inflammatory disorder comprising a step of administering the compound as defined by Formula I-XLII to a patient suffering from a cancer or inflammatory disorder.

The present invention also provides a use of the compound as defined by Formula I-XLII, preferably labelled or tagged, as an ADP-ribosyltransferase inhibitor in vitro. The present invention is thus directed to an in vitro method comprising a step contacting the compound as defined by Formula I-XLII, preferably labelled or tagged, with a sample suspected or known to comprise PARPs/TNKSs, in order to inhibit activity of the ADP-ribosyltransferase in the sample. Preferably, said method comprises a further step of contacting said compound with a control sample comprising an ADP-ribosyltransferase. In an embodiment, a label coupled to said compound is selected from the group consisting of fluorescence labels such as R6G, Cy3, Cy5, TAMRA, ROX, and FAM. In an embodiment, a tag coupled to said compound is biotin.

In an further embodiment, the present invention is also directed to an in vitro screening method for identifying inhibitors of an ADP-ribosyltransferase comprising:
a) contacting a candidate compound having a general formula

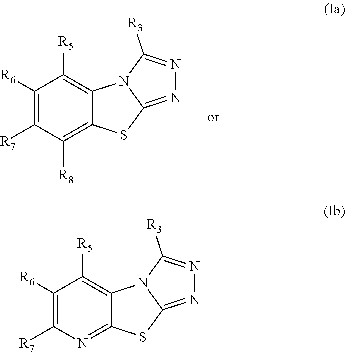

wherein:
R3, R5, R6, R7 and R8 are independently selected from the group consisting of: H, —NH₂, —COOH, —CN, —NO₂, —SH, —OH, F, Cl, Br, I, C₁₋₄ alkyl, aryl, C₁₋₄ alkyl aryl, C₁₋₄ alkyl halide, C₁₋₄ amino alkyl, aryl halide, C₁₋₄ alkyl aryl halide, alkoxy —OR, ester —OC(═O)R, amine —NHR, amide —NHC(═O)R, imine —(N=)R, NHSO₂(C₁₋₄ alkyl), and thioether —SR; wherein R is selected from the group consisting of: C₁₋₄ alkyl, aryl, C₁₋₄ alkyl aryl, C₁₋₄ alkylhalide, aryl halide, C₁₋₄ amino alkyl, C₃₋₆ cycloalkyl and C₁₋₄ alkyl aryl halide;
or a salt thereof;
with an ADP-ribosyltransferase;
b) measuring the level of enzymatic activity of the ADP-ribosyltransferase in the presence of said candidate compound;
c) selecting those candidate compounds which inhibit the ADP-ribosyltransferase.
   Preferably, the candidate compound selected in step c) can rescue cells overexpressing said ADP-ribosyltransferase. The candidate compound selected in step c) is tested to be a selective inhibitor of said ADP-ribosyltransferase.

Formulas I-XLII can be synthesized from commercial starting materials by known methods to those skilled in the art as exemplified in the following Experimental Section.

Unless otherwise stated, properties that have been experimentally measured or determined herein have been measured or determined at room temperature. Unless otherwise indicated, room temperature is 25° C.

Unless otherwise stated, properties that have been experimentally measured or determined herein have been measured or determined at atmospheric pressure.

As used herein, the terms “administering” or “administration” to a subject of a therapeutic agent, composition or compound as described herein includes any route of introducing or delivering to the subject the composition or compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “subject,” “individual,” “host,” and “patient,” are used interchangeably herein to refer to an animal being treated with one or more exemplary compounds as taught herein, including, but not limited to, simians, humans, avians, felines, canines, equines, rodents, bovines, porcines, ovines, caprines, mammalian farm animals, mammalian sport animals, and mammalian pets. A suitable subject for various embodiments can be any animal, including a human, that is suspected of having, has been diagnosed as having, or is at risk of developing a disease that can be ameliorated, treated or prevented by administration of one or more exemplary compounds as described herein.

As used herein, the term “treatment” or “treating” refers to administration of the compound of the invention to a subject, e.g., a mammal or human subject, for purposes which include not only complete cure, but also prophylaxis, amelioration, or alleviation of a disorder or symptoms related to a pathological condition. The therapeutic effect may be assessed by monitoring the symptoms of a patient, biomarkers in blood, a size of an injury or lesion, and/or or the length of survival of the patient.

It must also be noted that, as used in the specification and the appended claims, the singular forms “a,” “an” and “the” include plural referents unless otherwise specified.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

Having now generally described the invention, the same will be more readily understood by reference to the following Experimental Section, which is provided by way of illustration and is not intended as limiting.

### Experimental Section

### Materials and Methods

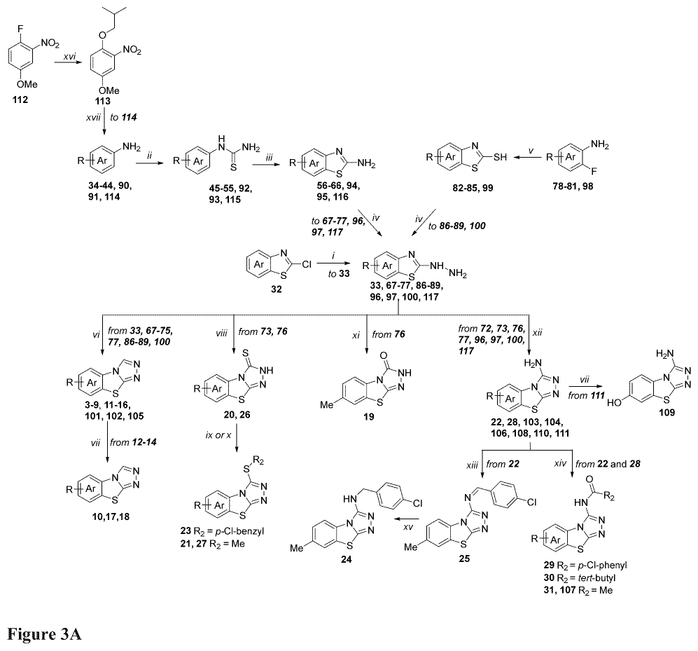

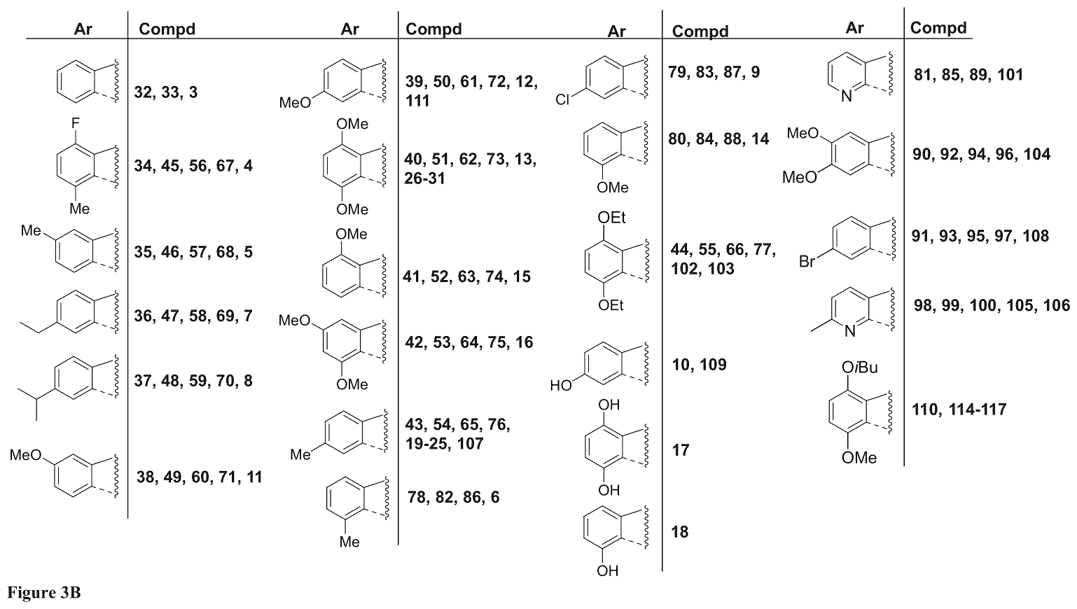

All starting materials, reagents, and solvents were purchased from common commercial suppliers and were used as such without further purification. Compounds 3, 9, 10, 12, 19, 20, 33, 45-54, 57-65, 68, 71, 72, 74, 76, 82-89, 92-97, 99, 105 were prepared as described in FIG. 3A. Compounds 32, 34-44, 78-81, 90, 91, 98, and 112 were commercially available. Organic solutions were dried over anhydrous Na₂SO₄ and concentrated with rotary evaporator at low pressure. All the reactions were routinely checked by thin-layer chromatography (TLC) on silica gel 60F254 (Merck) and visualized by using UV and iodine. Flash chromatography separations were carried out on Merck silica gel 60 (mesh 230-400) or by using automated Buchi Reveleris X2-UV with column FP Ecoflex Si 12 g. Yields were of purified products and were not optimized. ¹HNMR spectra were recorded at 400 MHz (Bruker Avance DRX-400), while ¹³CNMR spectra were recorded at 101 MHz (Bruker Avance DRX-400). Chemical shifts are given in ppm (δ) relative to TMS. Spectra were acquired at 298° K. Data processing was performed with standard Bruker software XwinNMR, and the spectral data are consistent with the assigned structures. Mating constant (J) are reported in Hz. The purity of the tested compounds was evaluated by HPLC analysis using Jasco LC-4000 instrument equipped with a UV-Visible Diode Array Jasco-MD4015 (Jasco Corporation, Tokyo, Japan), and XTerra MS C18 column, 5 μm×4.6 mm×150 mm (Waters Corporation, Massachusetts, USA). Chromatograms were analyzed by ChromNAV2.0 Chromatography Data System software. The purity of the compounds, performed at, 254 nm, at the, max of each compound and the absolute maximum of absorbance between 200 and 600 nm, was >95%. The peak retention time (ret. time) is given in minutes. High resolution mass detection was performed for some representative compounds and it was based on electrospray ionization (ESI) in positive polarity using Agilent 1290 Infinity System equipped with a MS detector Agilent 6540A Accurate Mass Q-TOF.
1-Isobutoxy-4-methoxy-2-nitrobenzene (113). Isobutyl alcohol (0.6 mL, 6.43 mmol) was added, under nitrogen atmosphere at 0° C., to a suspension of NaH (0.26 g, 6.43 mmol) in dry DMF. After 15 minutes, 1-fluoro-4-methoxy-2-nitrobenzene 112 (1 g, 5.84 mmol) was also added and the reaction mixture was stirred at 80° C. for 4 h. Then, it was poured into ice/water and extracted with EtOAc (×3), and the organic layers were washed with brine, dried over Na₂SO₄ and evaporated to dryness under reduced pressure to give 113 as an oil (0.74 g, 56%). ¹H NMR (400 MHz, DMSO-d₆) δ: 0.94 (6H, d, J=6.7 Hz, CH₃), 1.99 (1H, sett, CH), 3.78 (3H, s, OCH₃), 3.86 (2H, d, J=6.4 Hz, OCH₂), 7.18-7.29 (2H, m, aromatic H), 7.43-7.45 (1H, m, aromatic H).
(2-Isobutoxy-5-methoxyphenyl)amine (114). Zn (1.57 g, 24 mmol) and NH₄C1 (2.45 g, 40 mmol) were added to a solution of 113 (0.46 g, 2 mmol) in acetone/H₂O (50 mL, ratio 5:1). The reaction mixture was stirred at 60° C. for 3 h and, then, it was filtered over celite. The filtrate was removed under reduced pressure and CH₂Cl₂ was added, washed with NaHCO₃ saturated water, dried over Na₂SO₄ and evaporated to dryness under reduced pressure to give 114 as an oil (0.24 g, 62%). ¹H NMR (400 MHz, DMSO-d₆) δ: 0.99 (6H, d, J=6.2 Hz, CH₃), 1.98-1.99 (1H, m, CH), 3.61-3.63 (5H, m, OCH₂ and OCH₃), 4.70 (2H, bs, NH₂), 6.05 (1H, d, J=8.6 Hz, H3), 6.26 (1H, s, H6), 6.65 (1H, d, J=8.3 Hz, H4).
N-(2,5-diethoxyphenyl)thiourea (55). NH₄SCN (1.3 g, 16.56 mmol) was added to a solution of 2,5-diethoxy aniline 44 (1.00 g, 5.52 mmol) in H₂O (15 mL) and 12 N HCl (3 mL), and the reaction mixture was stirred at reflux for 16 h. Then, it was poured into ice/water and the formed precipitate was filtered to give 55 (0.53 g, 40% yield). ¹H NMR (400 MHz, DMSO-d₆) δ: 1.28-1.37 (6H, m, OCH₂CH₃), 3.93-3.95 (2H, m, OCH₂CH₃), 4.00-4.02 (2H, m, OCH₂CH₃), 6.64 (1H, d, J=7.8 Hz, H3), 6.92 (1H, d, J=8.9 Hz, H4), 7.56 (2H, bs, NH₂), 7.68 (1H, s, H6), 8.92 (1H, s, NH).
N-(2-isobutoxy-5-methoxyphenyl)thiourea (115). The title compound was synthesized following the same procedure as used for the synthesis of compound 55, starting from 114 in 38% yield as a solid, after trituration with Et₂O. ¹H NMR (400 MHz, DMSO-d₆) δ: 0.98 (6H, d, J=6.5 Hz, CH₃), 1.99-2.01 (1H, m, CH), 3.70-3.72 (5H, m, OCH₂ and OCH₃), 6.67 (1H, d, J=7.1 Hz, H3), 6.93 (1H, d, J=8.8 Hz, H4), 7.42 (1H, s, H6), 7.50 (2H, bs, NH₂), 8.87 (1H, bs, NH).
4-Fluoro-7-methyl-1,3-benzothiazol-2-amine (56). A solution of Br₂ (0.2 mL, 3.3 mmol) in CHCl₃ (5 mL) was slowly added to a suspension of 45 (0.6 g, 3.3 mmol) in CHCl₃ (13 mL), at 0° C. The reaction mixture was stirred at r.t. for 4 h and, then, a 10% solution of Na₂SO₃ in water was added to the mixture. CHCl₃ was removed under reduced pressure and NH₄₀H solution was added until the formation of a precipitate that was filtered, giving compound 56 (0.36 g, 66%). ¹H NMR (400 MHz, DMSO-d₆) δ: 2.46 (3H, s, CH₃), 6.75-6.78 (1H, m, aromatic CH), 6.92-7.00 (1H, m, aromatic CH), 7.67 (2H, bs, NH₂).
4,7-Diethoxy-1,3-benzothiazol-2-amine (66). The title compound was synthesized following the same procedure as used for the synthesis of compound 56, starting from 55 in 80% yield as a solid. ¹H NMR (400 MHz, DMSO-d₆) δ: 1.29-1.35 (6H, m, OCH₂CH₃), 4.02-4.10 (4H, m, OCH₂CH₃), 6.55 (1H, d, J=8.7 Hz, H5), 6.74 (1H, d, J=8.7 Hz, H6), 7.45 (2H, s, NH₂).
4-Isobutoxy-7-methoxy-1,3-benzothiazol-2-amine (116). The title compound was synthesized following the same procedure as used for the synthesis of compound 56, starting from 115 in 83% yield as a solid. ¹H NMR (400 MHz, DMSO-d₆) δ: 0.99 (6H, d, J=6.7 Hz, CH₃), 1.99 (1H, sett, CH), 3.78 (2H, d, J=6.5 Hz, OCH₂), 3.80 (3H, s, OCH₃), 6.55 (1H, d, J=7.8 Hz, H5), 6.75 (1H, d, J=7.8 Hz, H6), 7.52 (2H, bs, NH₂).
4-Fluoro-2-hydrazino-7-methyl-1,3-benzothiazole (67), General procedure (A) for the synthesis of hydrazinobenzothiazoles. Hydrazine hydrate (0.23 mL, 4.77 mmol) and CH₃COOH (0.12 mL, 2.1 mmol) were added to a suspension of 56 (0.29 g, 1.59 mmol) in ethylene glycol (8 mL), and the reaction mixture was stirred for 24 h at 125° C. Then, the mixture was poured into ice/water and a saturated solution of NaHCO₃ was added until pH=8, to give a precipitate that was filtered yielding 67 (0.1 g, 32%). ¹H NMR (400 MHz, DMSO-d₆) δ: 2.26 (3H, s, CH₃), 5.11 (2H, bs, NH₂), 6.72-6.76 (1H, m, aromatic H), 6.91-6.96 (1H, m, aromatic H), 9.09 (1H, bs, NH).
6-Ethyl-2-hydrazino-1,3-benzothiazole (69). The title compound was prepared according to the above general procedure A, starting from 58 (36 h) in 42% yield. ¹H NMR (400 MHz, DMSO-d₆) δ: 1.19 (3H, t, J=7.4 Hz, CH₂CH₃), 2.62 (2H, q, J=7.4 Hz, CH₂CH₃), 4.96 (2H, bs, NH₂), 7.04 (1H, d, J=7.6 Hz, H4), 7.23 (1H, d, J=7.9 Hz, H5), 7.51 (1H, s, H7), 8.86 (1H, bs, NH).
6-Ispropyl-2-hydrazino-1,3-benzothiazole (70). The title compound was prepared according to the above general procedure A, starting from 59 (30 h) in 52% yield. ¹H NMR (400 MHz, DMSO-d₆) δ: 1.21 (3H, d, J=2.1 Hz, CH₃), 1.22 (3H, d, J=2.1 Hz, CH₃), 2.87-2.91 (1H, m, CH), 4.97 (2H, bs, NH₂), 7.08 (1H, d, J=8.3 Hz, H4), 7.23 (1H, dd, J=1.9 and 8.2 Hz, H5), 7.55 (1H, s, H7), 8.88 (1H, bs, NH).
2-Hydrazino-4,7-dimethoxy-1,3-benzothiazole (73). The title compound was prepared according to the above general procedure A, starting from 62 (26 h) in 20% yield. ¹H NMR (400 MHz, DMSO-d₆) δ: 3.73 (3H, s, OCH₃), 3.77 (3H, s, OCH₃), 5.00 (2H, bs, NH₂), 6.48 (1H, d, J=8.6 Hz, H5), 6.70 (1H, d, J=8.6 Hz, H6), 8.86 (1H, bs, NH).
2-Hydrazino-5,7-dimethoxy-1,3-benzothiazole (75). The title compound was prepared according to the above general procedure A, starting from 64 (20 h) in 44% yield. ¹H NMR (400 MHz, DMSO-d₆) δ: 3.75 (3H, s, OCH₃), 3.87 (3H, s, OCH₃), 5.00 (2H, bs, NH₂), 6.27 (1H, s, H5), 6.56 (1H, s, H6), 8.98 (1H, bs, NH).
2-Hydrazino-4,7-Diethoxy-1,3-benzothiazole (77). The title compound was prepared according to the above general procedure A, starting from 66 (24 h) in 76% yield. ¹H NMR (400 MHz, DMSO-d₆) δ: 1.30-1.36 (6H, m, OCH₂CH₃), 4.03-4.11 (4H, m, OCH₂CH₃), 5.03 (2H, s, NH₂), 6.50-6.56 (1H, m, H5), 6.72-6.74 (1H, m, H6), 8.99 (1H, s, NH).
2-Hydrazino-5-methyl[1,3]thiazolo[5,4-b]pyridine (100). The title compound was prepared according to the above general procedure A, starting from 99 (48 h), using an excess of hydrazine hydrate (2 mL) and CH₃COOH (1 mL) in 25% yield, after purification by automatic column chromatography eluting with CH₃Cl:MeOH (98:2). ¹H NMR (400 MHz, DMSO-d₆) δ: 2.43 (3H, s, CH₃), 5.10 (2H, bs, NH₂), 7.08 (1H, d, J=8.0 Hz, H4), 7.50 (1H, d, J=8.0 Hz, H5), 9.19 (1H, bs, NH).
2-Hydrazino-4-isobutoxy-7-methoxy-1,3-benzothiazole (117). The title compound was prepared according to the above general procedure A, starting from 116 (36 h), in 57% yield. ¹H NMR (400 MHz, DMSO-d₆) δ: 0.97 (6H, s, CH₃), 1.96-1.98 (1H, m, CH), 3.33-3.36 (2H, m, OCH₂), 3.80 (3H, s, OCH₃), 5.05 (2H, bs, NH₂), 6.50-6.52 (1H, m, H5), 6.65-6.67 (1H, m, H6), 9.08 (1H, bs, NH).
5-Fluoro-8-methyl[1,2,4]triazolo[3,4-b][1,3]benzothiazole (4). General procedure (B) for the synthesis of [1,2,4]triazolo[3,4-b][1,3]benzothiazoles. A solution of 67 (0.13 g, 0.65 mmol) in formic acid (5 mL) was refluxed for 10 h. The reaction mixture was then poured into ice/water and the pH was neutralized using a saturated solution of NaHCO₃. The reaction mixture was extracted with EtOAc (×3) and the organic layers were washed with brine, dried over Na₂SO₄ and evaporated to dryness under reduced pressure to give a solid that was purified by flash chromatography eluting with CH₂Cl₂:MeOH (98:2), to give 4 (0.02 g, 13%). ¹H NMR (400 MHz, DMSO-d₆) δ: 2.37 (3H, s, CH₃), 7.31-7.32 (1H, m, H7), 7.43 (1H, t, J=8.3 Hz, H6), 9.43 (1H, s, H3). ¹³C NMR (101 MHz, DMSO-d₆) δ: 18.91, 114.06 (d, J=16.5 Hz), 117.63 (d, J=16.2 Hz), 127.89 (d, J=6.5 Hz), 130.21 (d, J=4.0 Hz), 133.54 (d, J=2.5 Hz), 138.15, 148.63 (d, J=245 Hz), 154.06. HPLC: CH₃CN/H₂O+0.1% FA (70:30), ret. time: 2.12 min, peak area: 100%.
6-Methyl[1,2,4]triazolo[3,4-b][1,3]benzothiazole (5). The title compound was prepared according to the above general procedure B starting from 68 (9 h) in 10% yield as a yellow solid, after purification by crystallization using cyclohexane/EtOAc (2:1). ¹H NMR (400 MHz, DMSO-d₆) δ: 2.41 (3H, s, CH₃), 7.28 (1H, d, J=8.2 Hz, H7), 7.87 (1H, d, J=8.2 Hz, H8), 7.92 (1H, s, H5), 9.55 (1H, s, H3). ¹³C NMR (101 MHz, DMSO-d₆) δ: 21.34, 115.50, 125.48, 127.99, 128.67, 129.35, 137.00, 137.41, 155.12. HPLC: CH₃CN/H₂O+0.1% FA (70:30), ret. time: 2.05 min, peak area: 99.21%.
8-Methyl[1,2,4]triazolo[3,4-b][1,3]benzothiazole (6). The title compound was prepared according to the above general procedure B starting from 86 (12 h) in 30% yield as a pink solid, after purification by flash chromatography eluting with CHCl₃:MeOH (95:5). ¹H NMR (400 MHz, DMSO-d₆) δ: 2.42 (3H, s, CH₃), 7.27-7.31 (1H, m, H7), 7.48 (1H, t, J=7.7 Hz, H6), 7.91-7.93 (1H, m, H5), 9.65 (1H, s, H3). ¹³C NMR (101 MHz, DMSO-d₆) δ: 19.70, 112.85, 127.55, 127.61, 129.18, 131.54, 134.71, 137.44, 154.07. HPLC: CH₃CN/H₂O+0.1% FA (70:30), ret. time: 2.06 min, peak area: 100%.
7-Ethyl[1,2,4]triazolo[3,4-b][1,3]benzothiazole (7). The title compound was prepared according to the above general procedure B starting from 69 (24 h) in 50% yield as a yellow solid, after purification by flash chromatography eluting with CHCl₃:MeOH (99:1). ¹H NMR (400 MHz, DMSO-d₆) δ: 1.23 (3H, t, J=7.5 Hz, CH₂CH₃), 2.73 (2H, q, J=7.6 Hz, CH₂CH₃), 7.44 (1H, dd, J=1.0 and 7.3 Hz, H6), 7.89 (1H, s, H8), 8.02 (1H, d, J=8.3 Hz, H5), 9.60 (1H, s, H3). ¹³C NMR (101 MHz, DMSO-d₆) δ: 16.18, 28.61, 115.13, 124.77, 127.33, 127.62, 132.13, 137.18, 143.33, 154.88. HPLC: CH₃CN/H₂O (65:35), ret. time: 2.81 min, peak area: 98.49%.
7-Isopropyl[1,2,4]triazolo[3,4-b][1,3]benzothiazole (8). The title compound was prepared according to the above general procedure B starting from 70 (24 h) in 58% yield as a white solid, after purification by flash chromatography eluting with CHCl₃:MeOH (99:1) and successive treatment with cyclohexane. ¹H NMR (400 MHz, DMSO-d₆) δ: 1.26 (6H, d, J=6.9 Hz, CH₃), 3.01-3.04 (1H, m, CH), 7.47-7.49 (1H, m, H6), 7.95 (1H, s, H8), 8.03 (1H, d, J=8.3 Hz, H5), 9.60 (1H, s, H3). ¹³C NMR (101 MHz, DMSO-d₆) δ: 24.44, 34.07, 115.15, 123.44, 126.05, 127.70, 132.16, 137.19, 147.99, 154.93. HPLC: CH₃CN/H₂O (65:35), ret. time: 3.50 min, peak area: 98.13%.
6-Methoxy[1,2,4]triazolo[3,4-b][1,3]benzothiazole (11). The title compound was prepared according to the above general procedure B starting from 71 (24 h) in 15% yield as a white solid, after purification by flash chromatography eluting with CH₂Cl₂:MeOH (98:2). ¹H NMR (400 MHz, DMSO-d₆) δ: 3.93 (3H, s, OCH₃), 8.01 (2H, s, aromatic H), 8.30 (1H, s, aromatic H), 9.56 (1H, s, H3). ¹³C NMR (101 MHz, DMSO-d₆) δ: 57.45, 100.22, 108.92, 123.75, 129.21, 129.73, 137.09, 155.21, 155.82. HPLC: CH₃CN/H₂O+0.1% FA (70:30), ret. time: 2.19 min, peak area: 99.72%.
5,8-Dimethoxy[1,2,4]triazolo[3,4-b][1,3]benzothiazole (13). The title compound was prepared according to the above general procedure B starting from 73 (12 h) in 25% yield as a pink solid, after purification by crystallization using cyclohexane/EtOAc (2:1). ¹H NMR (400 MHz, DMSO-d₆) δ: 3.89 (3H, s, OCH₃), 3.95 (3H, s, OCH₃), 7.04 (1H, d, J=9.0 Hz, H6), 7.15 (1H, d, J=9.0 Hz, H7), 9.29 (1H, s, H3). ¹³C NMR (101 MHz, DMSO-d₆) δ: 56.85, 57.12, 108.40, 110.55, 119.58, 119.97, 138.20, 142.52, 148.15, 154.51. HIRMS: m/z calcd for C₂₇H₂₈N₃O₄S 258.0313 [M+Na⁺], found 258.0310. HPLC: CH₃CN/H₂O+0.1% FA (70:30), ret. time: 2.15 min, peak area: 99.53%.
8-Methoxy[1,2,4]triazolo[3,4-b][1,3]benzothiazole (14). The title compound was prepared according to the above general procedure B starting from 88 (6 h) in 31% yield as a pink solid, after purification by flash chromatography eluting with CH₂Cl₂:MeOH (98:2). ¹H NMR (400 MHz, DMSO-d₆) δ: 3.95 (3H, s, OCH₃), 7.12 (1H, d, J=8.2 Hz, H7), 7.49-7.55 (1H, m, H6), 7.67 (1H, d, J=8.2 Hz, H5), 9.56 (1H, s, H3). ¹³C NMR (101 MHz, DMSO-d₆) δ: 56.87, 107.90, 108.81, 118.83, 129.02, 130.20, 137.42, 148.12, 154.79. HPLC: CH₃CN/H₂O+0.1% FA (70:30), ret. time: 1.70 min, peak area: 98.77%.
5-Methoxy[1,2,4]triazolo[3,4-b][1,3]benzothiazole (15). The title compound was prepared according to the above general procedure B starting from 74 (6 h) in 23% yield as a yellow solid, after purification by flash chromatography eluting with CH₂Cl₂:MeOH (98:2). ¹H NMR (400 MHz, DMSO-d₆) δ: 4.01 (1H, s, OCH₃), 7.22 (1H, d, J=6.7 Hz, H6), 7.42 (1H, t, J=8.1 Hz, H7), 7.55 (1H, d, J=8.2 Hz, H8), 9.34 (1H, s, H3). ¹³C NMR (101 MHz, DMSO-d₆) δ: 56.98, 109.94, 117.20, 119.23, 127.71, 132.88, 138.28, 148.28, 154.57. HPLC: CH₃CN/H₂O+0.1% FA (70:30), ret. time: 1.70 min, peak area: 99.47%. 6,8-Dimethoxy[1,2,4]triazolo[3,4-b][1,3]benzothiazole (16). The title compound was prepared according to the above general procedure B starting from 75 (15 h) in 10% yield as a white solid, after purification by flash chromatography eluting with CH₂Cl₂:MeOH (98:2). ¹H NMR (400 MHz, DMSO-d₆) δ: 3.88 (1H, s, OCH₃), 3.96 (1H, s, OCH₃), 6.76 (1H, s, H7), 7.46 (1H, s, H5), 9.58 (1H, s, H3). ¹³C NMR (101 MHz, DMSO-d₆) δ: 56.72, 57.07, 93.28, 97.86, 110.21, 130.48, 137.27, 155.35, 155.42, 161.11. HPLC: CH₃CN/H₂O (70:30), ret. time: 1.78 min, peak area: 97.88%.
[1,2,4]Triazolo[3′,4′:2,3][1,3]thiazolo[5,4-b]pyridine (101). The title compound was prepared according to the above general procedure B (24 h) starting from 89 in 20% yield as a yellowish solid, after purification by flash chromatography eluting with CHCl₃:MeOH (95:5). ¹H NMR (400 MHz, DMSO-d₆) δ: 7.67-7.68 (1H, m, H6), 8.52 (1H, d, J=8.1 Hz, H5), 8.58 (1H, d, J=5.0 Hz, H7), 9.70 (1H, s, H3). ¹³C NMR (101 MHz, DMSO-d₆) 6:122.64, 123.28, 125.99, 138.31, 148.13, 151.69, 154.11. HPLC: CH₃CN/H₂O (90:10), ret. time: 2.13 min, peak area: 98.64%.
5,8-Diethoxy[1,2,4]triazolo[3,4-b][1,3]benzothiazole (102). The title compound was prepared according to the above general procedure B (8 h) starting from 77 in 76% yield, as a white solid after purification by flash chromatography eluting with CH₂Cl₂:MeOH 95:5. ¹H NMR (400 MHz, DMSO-d₆) δ: 1.38 (3H, t, J=6.7 Hz, OCH₂CH₃), 1.47 (3H, t, J=7.6 Hz, OCH₂CH₃), 4.17-4.27 (4H, m, OCH₂CH₃), 7.06 (1H, d, J=8.9 Hz, H7), 7.17 (1H, d, J=8.9 Hz, H6), 9.23 (1H, s, H3). ¹³C NMR (101 MHz, DMSO-d₆) δ: 15.10, 15.14, 65.29, 65.69, 109.53, 110.25, 111.66, 119.97, 120.48, 138.16, 141.79, 147.56, 154.70. HPLC: CH₃CN/H₂O+0.1% DEA (90:10), ret. time: 2.64 min, peak area: 97.96%.
[1,2,4]Triazolo[3,4-b][1,3]benzothiazol-5,8-diol (17). A 1M solution of BBr₃ in CH₂Cl₂ (3.2 mL, 3.2 mmol) was added dropwise to a solution of 13 (0.15 g, 0.64 mmol) in dry CH₂Cl₂ at 0° C. and under nitrogen atmosphere. The reaction was stirred at r.t. overnight and, then, MeOH was added. The mixture was evaporated under reduced pressure to give a residue that was poured into ice/water, added of 6N HCl until pH 4, and extracted with EtOAc. The organic layers were washed with brine, dried over Na₂SO₄ and evaporated under reduced pressure, obtaining a solid that was purified by flash chromatography eluting with CH₂Cl₂:MeOH (95:5), yielding 17 as a purple solid (0.013 g, 10%). ¹H NMR (400 MHz, DMSO-d₆) δ: 6.79 (1H, d, J=8.7 Hz, H7), 6.89 (1H, d, J=8.8 Hz, H6), 9.24 (1H, s, H3), 10.29 (1H, s, OH), 10.43 (1H, s, OH). ¹³C NMR (101 MHz, DMSO-d₆) δ: 112.72, 114.60, 118.87, 119.17, 138.04, 139.41, 145.59, 154.59. HPLC: CH₃CN+0.1% FA/H2O+0.1% FA (50:50), ret. time: 1.89 min, peak area: 99.93%.
[1,2,4]Triazolo[3,4-b][1,3]benzothiazol-8-ol (18). The title compound was synthesized following the same procedure as used for the synthesis of compound 17 starting from 14 in 22% yield as a brown solid, after purification by flash chromatography eluting with CH₂Cl₂:MeOH (95:5). ¹H NMR (400 MHz, DMSO-d₆) δ: 6.96 (1H, d, J=8.2 Hz, H7), 7.43 (1H, t, J=8.1 Hz, H6), 7.59 (1H, d, J=8.1 Hz, H5), 9.62 (1H, s, H3), 11.14 (1H, s, OH). ¹³C NMR (101 MHz, DMSO-d₆) δ: 106.30, 112.68, 117.96, 128.84, 130.64, 137.40, 153.73, 154.89. HPLC: CH₃CN+0.1% FA/H₂O+0.1% FA (50:50), ret. time: 2.00 min, peak area: 99.12%.
7-Methyl-3-(methylthio)[1,2,4]triazolo[3,4-b][1,3]benzothiazole (21). K₂CO₃ (0.21 g, 1.5 mmol) and Mel (0.1 mL, 1 mmol) were added under nitrogen atmosphere to a solution of 20 (0.11 g, 0.5 mmol) in dry DMF (6 mL). The reaction mixture was stirred at 80° C. for 1.5 h and then poured into ice/water. The mixture was acidified with 2N HCl until pH 5 and the obtained precipitate was filtered and purified by crystallization using cyclohexane: EtOAc (2:1), obtaining 21 as a brown solid (0.03 g, 22%). ¹H NMR (400 MHz, DMSO-d₆) δ: 2.39 (3H, s, CH₃), 2.70 (3H, s, SCH₃), 7.37 (1H, d, J=8.2 Hz, H5), 7.83 (1H, s, H8), 7.86 (1H, d, J=8.3 Hz, H6). ¹³C NMR (101 MHz, DMSO-d₆) δ: 16.08, 21.26, 113.95, 125.79, 127.62, 128.25, 131.87, 136.63, 144.35, 156.11. HPLC: CH₃CN/H₂O+0.1% FA (70:30), ret. time: 2.15 min, peak area: 99.88%.
7-Methyl[1,2,4]triazolo[3,4-b][1,3]benzothiazol-3-amine (22). CNBr (0.47 g, 4.44 mmol) was added to a solution of 76 (0.53 g, 2.96 mmol) in MeOH (10 mL) and the reaction mixture was refluxed for 3.5 h. Then, it was poured into ice/water, a saturated solution of NaHCO₃ was added until pH 8 and the obtained precipitate was filtered and purified by flash chromatography eluting with CHCl₃:MeOH (95:5), obtaining 22 as a brown solid (0.06 g, 10%). ¹H NMR (400 MHz, DMSO-d₆) δ: 2.39 (3H, s, CH₃), 6.41 (2H, bs, NH₂), 7.30 (1H, dd, J=0.8 and 8.3 Hz, H6), 7.71 (1H, s, H8), 7.90 (1H, d, J=8.3 Hz, H5). ¹³C NMR (101 MHz, DMSO-d₆) δ: 21.31, 113.76, 125.43, 127.63, 127.84, 131.43, 135.63, 149.11, 150.90. HRMS: m/z calcd for C₉H₈N₄S 205.0550 [M+H⁺], found 205.0544. HPLC: CH₃CN/H₂O (70:30), ret. time: 1.61 min, peak area: 98.97%.
3-[(4-Chlorobenzyl)thio]-7-methyl[1,2,4]triazolo[3,4-b][1,3]benzothiazole (23). A suspension of 20 (0.12 g, 0.54 mmol) and KOH (0.03 g, 0.54 mmol) in absolute EtOH (8 mL) was refluxed for 30 minutes under nitrogen atmosphere. Then, p-chlorobenzyl chloride (0.09 g, 0.54 mmol) was added and the reaction mixture was stirred at reflux. After 4 h, EtOH was removed under reduced pressure to give a residue that was added of water and extracted with EtOAc (×3). The organic layers were washed with brine, dried over Na₂SO₄ and evaporated to dryness under reduced pressure to give a solid that was purified by crystallization using cyclohexane: EtOAc (3:1), to give 23 as a white solid (0.05 g, 27%). ¹H NMR (400 MHz, DMSO-d₆) δ: 2.36 (1H, s, CH₃), 4.39 (2H, s, Bz CH₂), 7.22-7.31 (5H, m, aromatic H), 7.80 (1H, s, H8), 7.84 (1H, d, J=8.3 Hz, H6). ¹³C NMR (101 MHz, DMSO-d₆) δ: 21.25, 37.72, 114.10, 125.61, 127.57, 128.02, 128.73, 131.19, 131.72, 132.53, 136.43, 136.62, 142.34, 156.55. HPLC: CH₃CN/H₂O+0.1% FA (70:30), ret. time: 3.32 min, peak area: 99.52%.
N-(4-chlorobenzyl)-7-methyl[1,2,4]triazolo[3,4-b][1,3]benzothiazol-3-amine (24). NaBH₄ (0.026 g, 0.69 mmol) was added to a suspension of 25 (0.15 g, 0.46 mmol) in EtOH (10 mL), at 0° C. and under nitrogen atmosphere. The reaction mixture was stirred overnight at r.t. and, then, it was poured into ice/water, saturated solution of NaHCO₃ was added until pH 8 furnishing a precipitate that was filtered and purified by flash chromatography eluting with CH₂Cl₂:MeOH (97:3), to give 24 as a green solid (0.03 g, 17%). ¹H NMR (400 MHz, DMSO-d₆) δ: 2.41 (3H, s, CH₃), 4.54 (2H, d, J=5.6 Hz, Bz CH₂), 7.23 (1H, bs, NH), 7.34 (1H, d, J=8.3 Hz, H5), 7.39 (2H, d, J=8.3 Hz, H3′ and H5′), 7.49 (2H, d, J=8.3 Hz, H2′ and H6′), 7.72 (1H, s, H8), 7.97 (1H, d, J=8.3 Hz, H6). ¹³C NMR (101 MHz, DMSO-d₆) δ: 21.13, 46.17, 113.61, 125.33, 127.45, 127.57, 128.47, 129.81, 131.24, 131.70, 135.59, 139.03, 149.76, 150.64. HPLC: CH₃CN/H₂O+0.1% FA (60:40), ret. time: 2.64 min, peak area: 99.11%.
N-[(1Z)-(4-chlorophenyl)methylene]-7-methyl[1,2,4]triazolo[3,4-b][1,3]benzothiazol-3-amine (25). p-TsOH (10 mg, 10% w/w) and p-chlorobenzaldehyde (0.07 g, 0.5 mmol) were added to a solution of 22 (0.10 g, 0.5 mmol) in dry benzene (20 mL). The reaction mixture was stirred at reflux by using a Dean-Stark apparatus for 16 h and, then, poured into ice/water. A saturated solution of NaHCO₃ was added until pH 8, the mixture was extracted with CH₂Cl₂ (×3) and the organic layers were washed with brine, dried over Na₂SO₄ and evaporated to dryness under reduced pressure. The obtained oil was purified by flash chromatography eluting with CH₂Cl₂:MeOH (95:5) and then treated with Et₂₀ to give 25 as a brown solid (0.02 g, 10%). ¹H NMR (400 MHz, DMSO-d₆) δ: 2.51 (1H, s, CH₃), 7.43 (1H, d, J=8.3 Hz, H5), 7.68 (2H, d, J=8.5 Hz, H3′ and H5′), 7.85 (1H, s, H8), 8.14 (1H, d, J=8.2 Hz, H6), 8.21 (2H, d, J=8.5 Hz, H2′ and H6′), 9.41 (1H, s, CH). ¹³C NMR (101 MHz, DMSO-d₆) δ: 21.55, 115.30, 125.72, 127.98, 128.94, 129.90, 132.03, 132.13, 134.24, 136.83, 138.51, 153.43, 155.14, 163.73. HRMS: m/z calcd for C₁₆H₁₁ClN₄S 327.04770 [M+H⁺], found 327.0472. HPLC: CH₃CN/H₂O+0.1% FA (60:40), ret. time: 5.80 min, peak area: 95.64%.
5,8-Dimethoxy[1,2,4]triazolo[3,4-b][1,3]benzothiazole-3(2H)-thione (26). KOH (0.09 g, 1.7 mmol) dissolved in few drops of H₂O was added to a suspension of 73 (0.38 g, 1.70 mmol) in EtOH (7 mL) and, then, CS₂ (0.5 mL, 8.5 mmol) was added. The reaction mixture was refluxed for 2 h and, then, EtOH was removed under reduced pressure and 2N HCl was added. The obtained precipitate was filtered, purified by flash chromatography eluting with cyclohexane:EtOAc (80:20) and treated with EtOH, to give 26 as a purple solid (0.08 g, 18%). ¹H NMR (400 MHz, DMSO-d₆) δ: 3.85 (3H, s, OCH₃), 3.93 (3H, s, OCH₃), 7.13 (1H, d, J=9.0 Hz, H6), 7.20 (1H, d, J=9.0 Hz, H7), 13.91 (1H, s, NH). ¹³C NMR (101 MHz, DMSO-d₆): 57.03, 57.82, 109.76, 114.61, 118.81, 122.65, 143.91, 148.03, 151.75, 163.65. HPLC: CH₃CN/H₂O (65:35), ret. time: 1.95 min, peak area: 96.90%.
5,8-Dimethoxy-3-(methylthio)-2,3-dihydro[1,2,4]triazolo[3,4-b][1,3]benzothiazole (27). The title compound was synthesized following the same procedure as used for the synthesis of compound 21 starting from 26 in 15% yield as a pink solid, after purification by flash chromatography eluting with cyclohexane:EtOAc (from 100:0 to 50:50). ¹H NMR (400 MHz, DMSO-d₆) δ: 2.61 (3H, s, SCH₃), 3.87 (3H, s, OCH₃), 3.89 (3H, s, OCH₃), 7.02 (1H, d, J=8.9 Hz, H6), 7.11 (1H, d, J=8.9 Hz, H7). ¹³C NMR (101 MHz, DMSO-d₆) δ: 15.79, 56.95, 57.07, 109.05, 111.58, 120.07, 120.71, 142.13, 146.93, 148.30, 156.33. HPLC: CH₃CN/H₂O (60:40), ret. time: 2.23 min, peak area: 95.80%.
5,8-Dimethoxy[1,2,4]triazolo[3,4-b][1,3]benzothiazol-3-amine (28). The title compound was synthesized following the same procedure as used for the synthesis of compound 22 starting from 73 in 12% yield as a pink solid, after purification by flash chromatography eluting with CHCl₃: MeOH (95:5) and successive treatment by EtOH. ¹H NMR (400 MHz, DMSO-d₆) δ: 3.90 (3H, s, OCH₃), 3.99 (3H, s, OCH₃), 6.46 (2H, bs, NH₂), 7.02 (1H, d, J=9.0 Hz, H6), 7.16 (1H, d, J=9.0 Hz, H7). ¹³C NMR (101 MHz, DMSO-d₆) δ: 57.00, 57.78, 108.36, 111.50, 120.03, 120.15, 141.48, 148.45, 148.55, 150.67. HRMS: m/z calcd for C₁₀H₁₀N₄O₂S 251.0590 [M+H⁺], found 251.0584. HPLC: CH₃CN/H₂O (70:30), ret. time: 1.64 min, peak area: 97.78%.
5,8-Diethoxy[1,2,4]triazolo[3,4-b][1,3]benzothiazol-3-amine (103). The title compound was synthesized following the same procedure as used for the synthesis of compound 22 starting from 77 (8 h) in 20% yield as brown solid after purification by flash chromatography eluting with CHCl₃: MeOH 97: 3. ¹H NMR (400 MHz, DMSO-d₆) δ: 1.36 (3H, t, J=6.9 Hz, OCH₂CH₃), 1.42 (3H, t, J=6.9 Hz, OCH₂CH₃), 4.17 (2H, q, J=6.9 Hz, OCH₂CH₃), 4.26 (2H, q, J=6.9 Hz, OCH₂CH₃), 6.54 (2H, bs, NH₂), 7.00 (1H, d, J=9.0 Hz, H7), 7.14 (1H, d, J=9.0 Hz, H6). ¹³C NMR (101 MHz, DMSO-d₆) δ: 14.78, 15.13, 65.27, 66.56, 109.32, 112.50, 120.40, 120.41, 140.43, 147.85, 148.48, 150.58. HPLC: CH₃CN/H2O+0.1% DEA (90:10), ret. time: 2.63 min, peak area: 98.05%.
4-Chloro-N-(5,8-dimethoxy-2,3-dihydro[1,2,4]triazolo[3,4-b][1,3]benzothiazol-3-yl)benzamide (29). Et₃N (0.16 mL, 1.2 mmol) and p-chlorobenzoyl chloride (0.13 mL, 1.04 mmol) were added to a solution of 28 (0.22 g, 0.8 mmol) in dry DMF (6 mL) under nitrogen atmosphere. The reaction mixture was stirred for 2 h at 80° C. and, then, it was poured into ice/water obtaining a precipitate that was filtered and purified by flash chromatography eluting with CHCl₃:MeOH (95:5), to give 29 as a purple solid (0.03 g, 10%). ¹H NMR (400 MHz, DMSO-d₆) δ: 3.39 (3H, s, OCH₃), 3.95 (3H, s, OCH₃), 7.09-7.15 (2H, m, H6 and H7), 7.71 (2H, d, J=6.7 Hz, H3′ and H5′), 8.11 (2H, d, J=6.7 Hz, H2′ and H6′). ¹³C NMR (101 MHz, DMSO-d₆) δ: 57.02, 57.14, 109.15, 111.33, 120.10, 120.38, 129.30, 130.30, 131.38, 137.90, 142.56, 142.92, 148.01, 154.93, 166.58. HRMS: m/z calcd for C₁₇H₁₃ClN₄O₃S 389.048 [M+H⁺], found 389.047. HPLC: CH₃CN/H₂O+0.1% FA (60:40), ret. time: 2.17 min, peak area: 97.06%.
N-(5,8-dimethoxy[1,2,4]triazolo[3,4-b][1,3]benzothiazol-3-yl)-2,2-dimethylpropanamide (30). The title compound was synthesized following the same procedure as used for the synthesis of compound 29, starting from 28 and using trimethylacetyl chloride (0.11 mL, 0.88 mmol) and dry toluene as solvent at 110° C., overnight. After purification by flash chromatography eluting with CHCl₃:MeOH (97:3), compound 30 was obtained as a grey solid (0.06 g, 20%). ¹H NMR (400 MHz, DMSO-d₆) δ: 1.29 (9H, s, CH₃) 3.89 (3H, s, OCH₃), 3.95 (3H, s, OCH₃), 7.13 (1H, d, J=9.1 Hz, H7), 7.22 (1H, d, J=9.1 Hz, H6), 10.14 (1H, s, NH). ¹³C NMR (101 MHz, DMSO-d₆) δ: 27.68, 57.18, 57.99, 109.25, 112.11, 120.40, 120.55, 143.45, 143.49, 148.22, 154.51, 156.13, 179.03. HPLC: CH₃CN/H₂O+0.1% FA (70:30), ret. time: 1.77 min, peak area: 99.76%.
N-(5,8-dimethoxy[1,2,4]triazolo[3,4-b][1,3]benzothiazol-3-yl)acetamide (31). The title compound was synthesized following the same procedure as used for the synthesis of compound 29, starting from 28 and using acetyl chloride (0.63 mL, 0.88 mmol) and dry toluene as solvent at 110° C., overnight. The reaction mixture was extracted with EtOAc, the organic layers were washed with brine, dried over Na₂SO₄ and evaporated under reduced pressure, obtaining an oil that was purified by flash chromatography eluting with CHCl₃:MeOH (97:3), to give 31 as a pink solid (0.05 g, 20%). ¹H NMR (400 MHz, DMSO-d₆) δ: 2.19 (3H, s, CH₃), 3.87 (3H, s, OCH₃), 3.90 (3H, s, OCH₃), 6.81 (1H, d, J=8.7 Hz, H7), 6.92 (1H, d, J=8.6 Hz, H6), 12.49 (1H, s, NH). ¹³C NMR (101 MHz, DMSO-d₆) δ: 23.22, 56.45, 56.88, 104.96, 108.99, 121.28, 139.95, 140.12, 146.97, 148.23, 157.34, 169.78. HPLC: CH₃CN/H₂O+0.1% FA (70:30), ret. time: 1.80 min, peak area: 95.05%.
6,7-Dimethoxy[1,2,4]triazolo[3,4-b][1,3]benzothiazol-3-amine (104). The title compound was synthesized following the same procedure as used for the synthesis of compound 22 starting from 96 in 10% yield as a pink solid, after purification by flash chromatography eluting with CHCl₃: MeOH 95:5. ¹H NMR (400 MHz, DMSO-d₆) δ: 3.80 (3H, s, OCH₃), 3.88 (3H, s, OCH₃), 6.47 (2H, s, NH₂), 7.56 (1H, s, H5), 7.59 (1H, s, H8). ¹³C NMR (101 MHz, DMSO-d₆) δ: 56.59, 26.89, 99.10, 108.41, 121.60, 123.42, 147.49, 148.65, 149.82, 159.39. HPLC: CH₃CN/H₂O (80:20), ret. time: 3.35 min, peak area: 99.47%.
7-Methyl[1,2,4]triazolo[3′,4′:2,3][1,3]thiazolo[5,4-b]pyridin-3-amine (106). The title compound was synthesized following the same procedure as used for the synthesis of compound 22 starting from 100 in 15% yield as a white solid, after purification by flash chromatography eluting with CHCl₃: MeOH 95:5. ¹H NMR (400 MHz, DMSO-d₆) δ: 2.50 (3H, s, CH₃), 6.59 (2H, bs, NH₂), 7.41 (1H, d, J=8.3 Hz, H5), 8.29 (1H, d, J=8.3 Hz, H7). ¹³C NMR (101 MHz, DMSO-d₆) δ: 23.97, 121.17, 121.43, 124.14, 145.43, 151.51, 152.99, 155.73. HPLC: CH₃CN/H₂O (80:20), ret. time: 3.31 min, peak area: 99.45%.
N-(7-methyl[1,2,4]triazolo[3,4-b][1,3]benzothiazol-3-yl)acetamide (107). The title compound was synthesized following the same procedure as used for the synthesis of compound 30, starting from 22 in 11% yield as a grey solid, after purification by flash chromatography eluting with CHCl₃:MeOH (98:2). ¹H NMR (400 MHz, DMSO-d₆) δ: 2.25 (3H, s, CH₃), 2.42 (3H, s, CH₃), 7.35 (1H, bs, H5), 7.45 (1H, bs, H6), 7.84 (1H, s, H8), 11.05 (1H, s, NH). ¹³C NMR (101 MHz, DMSO-d₆) δ: 21.44, 23.20, 115.33, 125.50, 127.57, 128.24, 131.71, 136.82, 142.17, 154.66, 171.45. HPLC: CH₃CN/H₂O+0.1% FA (35:65), ret. time: 3.59 min, peak area: 96.08%.
7-Bromo[1,2,4]triazolo[3,4-b][1,3]benzothiazol-3-amine (108). The title compound was synthesized following the same procedure as used for the synthesis of compound 22 starting from 97 in 25% yield as a white solid, after purification by flash chromatography eluting with CHCl₃: MeOH (95:5). ¹H NMR (400 MHz, DMSO-d₆) δ: 6.50 (2H, bs, NH₂), 7.68 (1H, d, J=8.5 Hz, H6), 7.94 (1H, d, J=8.4 Hz, H5), 8.23 (1H, s, H8). ¹³C NMR (101 MHz, DMSO-d₆) δ: 115.63, 117.71, 127.92, 129.28, 129.71, 133.89, 149.21, 151.02. HPLC: CH₃CN/H₂O (80:20), ret. time: 3.48 min, peak area: 99.48%.
3-Amino[1,2,4]triazolo[3,4-b][1,3]benzothiazol-7-ol (109). The title compound was synthesized following the same procedure as used for the synthesis of compound 17 starting from 111 in 15% yield as a brown solid, after purification by flash chromatography eluting with CH₂Cl₂:MeOH (90:10). ¹H NMR (400 MHz, DMSO-d₆) δ: 6.30 (2H, bs, NH₂), 6.85 (1H, dd, J=8.8 and 2.4 Hz, H6), 7.26 (1H, d, J=2.4 Hz, H8), 7.81 (1H, d, J=8.8 Hz, H5), 9.91 (1H, s, OH). ¹³C NMR (101 MHz, DMSO-d₆) δ: 110.46, 112.95, 113.63, 121.60, 131.54, 147.87, 149.45, 153.48. HPLC: CH₃CN/H₂O+0.1% FA (0 to 100%), ret. time: 5.14 min, peak area: 96.47%.
7-Methoxy[1,2,4]triazolo[3,4-b][1,3]benzothiazol-3-amine (111). The title compound was synthesized following the same procedure as used for the synthesis of compound 22 starting from 72 in 55% yield as a brown solid. ¹H NMR (400 MHz, DMSO-d₆) δ: 3.81 (3H, s, OCH₃), 6.37 (2H, bs, NH₂), 7.07 (1H, d, J=8.2 Hz, H6), 7.61 (1H, s, H8), 7.91 (1H, d, J=8.7 Hz, H5).
5-Isobutoxy-8-methoxy[1,2,4]triazolo[3,4-b][1,3]benzothiazol-3-amine (110). The title compound was synthesized following the same procedure as used for the synthesis of compound 22 starting from 117 in 20% yield as a yellowish solid, after purification by flash chromatography eluting with CHCl₃:MeOH (95:5). ¹H NMR (400 MHz, DMSO-d₆) δ: 1.03 (6H, d, J=6.7 Hz, CH₃), 2.10 (1H, sett, CH), 3.91 (3H, s, OCH₃), 4.02 (2H, d, J=Hz, OCH₂), 6.59 (2H, bs, NH₂), 7.01 (1H, d, J=9.1 Hz, H6), 7.17 (1H, d, J=9.1 Hz, H7). ¹³C NMR (101 MHz, DMSO-d₆) δ: 19.50, 27.99, 56.94, 76.85, 108.34, 112.16, 120.04, 120.13, 140.71, 148.40, 148.43, 150.44. HPLC: CH₃CN/H₂O+0.1% FA (0 to 100%), ret. time: 7.82 min, peak area: 98.37%.
Protein production. The proteins referred to in Tables 1-3 were produced as described earlier (Venkannagari et al., 2016). Dockerin constructs to allow enhanced activity assays for catalytic fragment mono-ARTs PARP7 (residues 448-657), PARP8 (611-844), PARP10 (809-1016), PARP11 (116-338), PARP12 (489-701), PARP14 (1535-1801) and PARP15 (481-678) (Table 4) were produced in E. coli with an N-terminal MBP and a C-terminal dockerin domain from Hungateiclostridium thermocellum Cel48s and purified in a similar manner.
Activity assay. Inhibition experiments were performed using a homogenous assay measuring NAD⁺ consumption (Narwal et al, 2012; Venkannagari et al, 2013). Reactions were carried out in quadruplicates and IC₅₀ curves were fitted using sigmoidal dose response curve (four variables) in GraphPad Prism version 8.02. For the compounds showing <1 μM potency the experiment was repeated three times and pIC₅₀±SEM was calculated. Assay conditions for PARP2, TNKS2, PARP10 and PARP15 (Tables 1-3) are recently reported (Maksimainen et al, 2021). The proximity enhanced mono-ART essays (Table 4, PARP7-15) were all carried out in the same buffer (50 mM sodium phosphate pH 7.0). The proximity enhanced activity was achieved by preparing a complex of multiple enzyme-dockerin proteins with C8 scaffolding (Galera-Part et al. 2018).

### Results

With the exception of compounds 1 and 2, which are commercially available, all the compounds were synthesized as shown in FIG. 3. Triazobenzothiazole target compounds 3-18, 101,102 and 105 variously functionalized on the benzene ring and 19-31, 103, 104, and 106-110 functionalized at the C-3 position, were prepared from the key intermediates 2-hydrazinobenzothiazoles 33, 67-77, 86-89, 96, 97, 100, and 117 obtained through three different synthetic pathways.
The unsubstituted hydrazinobenzothiazole 33 was obtained starting from 2-chlorobenzothiazole 32 by reaction with hydrazine hydrate in EtOH with a conversion of 98%.
Most of the 2-hydrazinobenzothiazoles (intermediates 67-77, 96, 97, and 117) were instead synthesized starting from the properly substituted anilines 34-44, 90, 91 and 114. Aniline 114 was, in turn, obtained from 2-fluoro-5-methoxy nitrobenzene 112 through nucleophilic substitution with isobutyl alcohol in the presence of NaH in dry DMF to obtain 113, which was then reduced into aniline 114 with NH₄Cl and Zn. Anilines 34-44, 90, 91 and 114 were converted into the corresponding arylthiourea derivatives 45-55, 92, 93, and 115 by reaction with NH₄SCN in acid H₂O at reflux. The successive oxidative cyclization of 45-55, 92, 93, 115 using Br₂ gave the corresponding 2-aminobenzothiazoles 56-66, 94, 95, and 116, which were then treated with hydrazine hydrate to give 2-hydrazinobenzothiazole derivatives 67-77, 96, 97, and 117. On the other hand, 2-hydrazinobenzothiazoles 86-88 and 2-hydrazinothiazolopyridines 89 and 100 were prepared starting from the 2-mercaptothiazoles 82-85, and 99, in turn obtained through a double nucleophilic substitution of properly substituted 2-fluoroanilines 78-80 and 3-amino-2-fluoropyridines 81 and 98 with potassium ethyl xanthogenate in dry DMF.
By reacting compounds 33, 67-75, 77, 86-89 and 100 with refluxed formic acid in excess, the tricyclic compounds 3-9, 11-16, 101, 102 and 105 were obtained with 10-58% yields. The methoxy derivatives 12-14 were further elaborated into the corresponding hydroxyl derivatives 10, 17 and 18 by using BBr₃.
Starting from 4,7-dimethoxy derivative 73 and 6-methyl derivative 76, by using CS₂ in EtOH, benzothiazole-3-thiones 26 and 20 were obtained, respectively. S-alkylation of methyl derivative 20 with Mel, K₂CO₃ in dry DMF and p-chlorobenzyl chloride in EtOH gave the corresponding derivatives 21 and 23, while, under the same conditions, 5,8-dimethoxy derivative 26 gave thiomethyl derivative 27. By treating 6-methyl derivative 76 with urea in neat condition and at the fusion temperature, benzothiazol-3-one 19 was obtained.
Finally, the reaction of 72, 73, 76, 77, 96, 97, 100, and 116 with CNBr furnished 3-aminobenzothiazole derivatives 22, 28, 103, 104, 106, 108, and 110 and the intermediate 111. The successive de-O-methylation of 111 using BBr₃ in dry CH₂Cl₂ gave the target compound 109. 3-Amino-7-methyl derivative 22 was successively condensed with p-chlorobenzaldehyde to give imine derivative 25, which was then reduced to amine derivative 24 with NaBH₄. Amidation of dimethoxy derivative 28 with p-chlorobenzoyl chloride in the presence of Et₃N in dry DMF gave compound 29. The reaction of 28 with trimethylacetyl chloride in the presence of Et₃N in dry toluene yielded compound 30, while the reaction of 28 and 22 with acetyl chloride in the same conditions furnished derivatives 31 and 107, respectively.
OUL40 scaffold. Our studies on compound OUL40 (1) revealed the potency of the compound scaffold by showing reasonable PARP enzyme inhibition even in the absence of the typical benzamide moiety. Importantly, no PARP inhibitor based on this tricyclic scaffold has been reported until now. Offering many options for substitutions, we were encouraged to study the scaffold in more detail.
At first, we were interested if small modifications would lead to any significant selectivity changes between mono- and poly-ARTs and to specific inhibition for individual ART subfamily. Iterative medicinal chemistry cycles were performed with a first set of compounds that emerged by working on benzene ring, where the methyl group of 1 was deleted (3), moved from C-7 to C-6 and C-8 positions (5 and 6), and replaced by bulkier groups as in compounds 7-9. Disubstituted derivatives 2 and 4 were also contemplated (Table 1). Further derivatives were characterized by the presence of monomethoxy (11, 12, 14, 15), dimethoxy (13 and 16) monohydroxy (10 and 18), and dihydroxy (17) groups, decorating the benzene ring (Table 2).
Subsequent biochemical and structural analyses described later suggested that the C-3 functionalization of the triazole ring could improve selectivity. Therefore, additional compounds were prepared by placing a heteroatom, oxygen, sulfur or nitrogen (19, 20, 22, 26, and 28) in this position, which was also derivatized as in compounds 21, 23-25, 27, 29-31, while maintaining a 7-methyl or a 5,8-dimethoxy substituent in the benzene ring (Table 3).
OUL40 (1) analogues: biochemical analysis and structural studies
All the synthesized TBTs were initially tested against representative members of the PARP family: two poly-ARTs PARP2 and TNKS2, and two mono-ARTs PARP10, and PARP15. The latter were selected based on availability of a robust cell based readout for target engagement and for a similarly robust crystal system of PARP15 to study compound binding modes experimentally. In addition, all the analogs were also routinely tested for the toxicity using the WST-1 assay, which showed that only the dihydroxy derivative 17 had dose dependent toxicity.
We first tested the effect of small alkyl groups and halogens on the benzene ring (Table 1). The only commercially available compound 2 had an additional methyl substituent and it showed improved potency against all the tested PARPs. The removal of the methyl group on the other hand reduced the potency of 3 especially against PARP10 and PARP15 (IC₅₀>10 μM) indicating that an electron donating hydrophobic substituent would be important for potency towards mono-ARTs. Changing of the methyl group to other positions did not have major effect on the potency (4-6) and all the compounds maintained μM potencies for tested enzymes. Compound 5 having the methyl in the C-6 position, however, showed higher potency against PARP2 and TNKS2. The TNKS2 crystal structure in complex with 5 revealed that the 6-methyl pushed Tyr1050 to different conformation and provided the additional interaction explaining the poly-ART selectivity. When C-7 methyl found in 1 was extended to a larger alkyl or substituted by a chlorine (7-9), it did not provide improvements to inhibition potency. Compound 9 with C-7 chlorine substituent also maintained a micromolar activity only against PARP2 and PARP15. The small substituent changes did not result in significant structural changes as observed with the PARP15 crystal structures in complex with 2, 8 and 9 as they showed highly similar binding modes compared to 1.
Next, we tested effects of hydroxyl and methoxy groups while keeping the general hydrophobic nature of the scaffold (Table 2). Interestingly, the presence of a C-7 hydroxyl made 10 very potent and specific for poly-ARTs with ten-fold selectivity for PARP2 over TNKS2 (IC₅₀ of 44 nM versus 370 nM).
Compounds 11-18 are in general active on all the enzymes tested (Table 2). In contrast to the hydroxyl of 10, the replacement of the methyl group of 1 with a methoxy gave compound 12 endowed with a similar profile with a similar binding mode. Dimethoxy derivative 13 showed submicromolar potency against PARP10 (IC₅₀=490 nM) and the corresponding di-hydroxyl analogue 17 expanded the nanomolar potency also against PARP15 and PARP2. This did not provide us the poly-ART vs. mono-ART selectivity, but the presence of an 8-methoxy group in compound 14 imparted selectivity against mono-ARTs PARP10 and PARP15.
We hypothesized that the plasticity would allow compounds 13 and 15 to inhibit multiple PARPs as the compound activities were still at a micromolar level against PARP2 and TNKS2 (Table 2). Therefore, we decided to add an anchor point to C-3 position in order to fix the compound orientation in the binding pocket. We tested multiple substituents at the C-3 position while preserving a methyl at C-7 or two methoxy groups at C-5 and C-8 as in 1 and 13, respectively. Compounds having a carbonyl (19) or small sulfur groups (20 and 21) integrated to the scaffold 1 emerged as selective against PARP10 with micromolar activities. A more interesting compound was achieved when using an amino group as C-3 substituent that gave 22 showing nanomolar activity against PARP10 (IC₅₀=180 nM) and PARP15 (IC₅₀=300 nM) with a clear selectivity against the mono-ARTs over the poly-ARTs PARP2 and TNKS2, which were inhibited only with micromolar potency (IC₅₀=1.6 μM and 5.7 μM, respectively).
We also extended the thiol and amino groups with a longer substituent but, independently by the heteroatom, this caused loss of activity (23-25). Only imine derivative 25 was active against PARP2 (IC₅₀=2.9 μM) and PARP15 (IC₅₀=7.9 μM) (Table 3). Pyridine derivative 101 showed only weak inhibition of PARP10 so it was not analyzed further. Regarding the C-3 substituted dimethoxy analogs, the inclusion of a thiol group to the scaffold 13 determined its loss of activity while 27 having a thiomethyl group recovered a modest selectivity against PARP2 (IC₅₀=8.4 μM). The presence of a 3-amino group imparted to 28 nanomolar activities against PARP10 (IC₅₀=120 nM) and PARP15 (IC₅₀=120 nM), but with significantly higher selectivity for mono-ADP-ribosylating enzymes in comparison a strict analog 22 (Table 3). The PARP15 crystal structure in complex with 28 showed highly similar binding mode as 13. To extend the anchor and potentially to improve even more the selectivity, we designed amino substituted analogs. Various amide derivatives were prepared, but in the presence of both either longer or shorter substituents, only very weak activity was observed against some enzymes (Table 3).
Extending the methoxy groups of 13 and 28 to create diethoxy analogs 102 and 103 was tolerated and resulted in potent PARP10 inhibitors (Table 3).

### Specificity Evaluation

The 7-hydroxy derivative 10, and the 3-amino derivatives 22 and 28 emerged as the most interesting compounds of the work as they were both potent and showed selectivity either towards mono- or poly-ARTs. We therefore profiled them against a larger panel of active PARPs (Table 4) with an improved assay method.
Compound 10 showed inhibition of poly-ARTs PARP1-2 and TNKS1-2 and also μM IC₅₀ values for active site glutamate containing mono-ARTs PARP3-4. This is consistent with the crystal structures where the hydroxyl of 10 forms a hydrogen bond with this glutamate residue. It shows interesting selectivity towards PARP2 even over highly similar PARP1 (13-fold) as well as tankyrases (8-fold).
Using the improved assay, it was discovered that both the 3-aminoderivatives 22 and 28 were potent inhibitors of multiple human mono-ARTs (Table 4). While 22 still inhibited multiple poly-ARTs at low μM concentration, 28 was overall more selective for mono-ARTs in agreement with our initial assessment. Compound 28 showed the best potency measured as it inhibited PARP10 with an IC₅₀ of 7.8 nM making it the best PARP10 inhibitor described to date. Additionally, 28 inhibited PARP7, PARP11, PARP12, PARP14 and PARP15 at low nanomolar potencies. Notably, no inhibitors of PARP12 has been described earlier.

TABLE 1
Profiling of compound 1 and the initial analogs. IC₅₀ (pIC₅₀ ± SEM) values.

| | 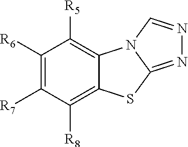 | | | | | | | | |
|---|---|---|---|---|---|---|---|---|---|
| Formula | ID | R₅ | R₆ | R₇ | R₈ | PARP2 | TNKS2 | PARP10 | PARP15 |
| V | 1* | —H | —H | —CH₃ | —H | 4.0 μM | 5.3 μM | 3.2 μM | 3.2 μM |
| VI | 2^{#} | —CH₃ | —H | —CH₃ | —H | 1.3 μM | 1.2 μM | 1.7 μM | 0.78 μM |
| | | | | | | | | | (6.11 ± 0.10) |
| VII | 3 | —H | —H | —H | —H | 5.3 μM | 2.5 μM | >10 μM | >10 μM |
| VIII | 4 | —F | —H | —H | —CH₃ | 6.9 μM | 5.4 μM | 5 μM | 6.9 μM |
| IX | 5 | —H | —CH₃ | —H | —H | 1.5 μM | 0.95 μM | 10 μM | 6.6 μM |
| | | | | | | | (6.02 ± 0.13) | | |
| X | 6 | —H | —H | —H | —CH₃ | 8.3 μM | 3.8 μM | 3.4 μM | 7.1 μM |
| XI | 7 | —H | —H | —CH₂CH₃ | —H | 3.9 μM | 5.3 μM | 4.9 μM | 2.0 μM |
| | | | | | | | | (5.31 ± 0.10) | (5.70 ± 0.04) |
| XII | 8 | —H | —H | —CH(CH₃)₂ | —H | 4.5 μM | 9.5 μM | 5.5 μM | 3.9 μM |
| | | | | | | | | (5.26 ± 0.08) | (5.41 ± 0.13) |
| XIII | 9 | —H | —H | —Cl | —H | 2.4 μM | 10 μM | >10 μM | 7.0 μM |

*Purchased from NCI DTP repository
^{#}Purchased from Specs.
>denotes less than 50% inhibition in the reported highest concentration and
>>denotes no inhibition at the reported concentration.
Compound 1 showed IC₅₀ of 1.2 0 μM against PARP14.

TABLE 2
Activities of methoxy and hydroxyl substituted analogs of compound 1. IC₅₀
(pIC₅₀ ± SEM) values.

| | 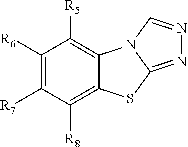 | | | | | | | | |
|---|---|---|---|---|---|---|---|---|---|
| Formula | ID | R₅ | R₆ | R₇ | R₈ | PARP2 | TNKS2 | PARP10 | PARP15 |
| IV | 10 | —H | —H | —OH | —H | 0.044 μM | 0.37 μM | >10 μM | >10 μM |
| | | | | | | (7.44 ± 0.12) | (6.43 ± 0.13) | | |
| XIV | 11 | —H | —OCH₃ | —H | —H | 4.7 μM | 1.0 μM | 1.8 μM | 7.1 μM |
| XV | 12 | —H | —H | —OCH₃ | —H | 8.8 μM | 5.5 μM | 6.5 μM | 4.0 μM |
| XVI | 13 | —OCH₃ | —H | —H | —OCH₃ | 9.2 μM | 4.0 μM | 0.49 μM | 1.6 μM |
| | | | | | | | | (6.31 ± 0.22) | |
| XVIII | 14 | —H | —H | —H | —OCH₃ | >10 μM | 24 μM | 1.9 μM | 1.2 μM |
| | | | | | | | | (5.71 ± 0.04) | (5.93 ± 0.16) |
| XVII | 15 | —OCH₃ | —H | —H | —H | 2.1 μM | 1.0 μM | 5.1 μM | 2.1 μM |
| | | | | | | | | (5.30 ± 0.15) | (5.67 ± 0.03) |
| XIX | 16 | —H | —OCH₃ | —H | —OCH₃ | 20 μM | 7.6 μM | 1.1 μM | 1.6 μM |
| XXIX | 17 | —OH | —H | —H | —OH | 0.24 μM | 3.1 μM | 0.32 μM | 0.29 μM |
| | | | | | | | | (6.50 ± 0.04) | (6.54 ± 0.05) |
| XXX | 18 | —H | —H | —H | —OH | 29 μM | 17 μM | >10 μM | 4.70 μM |
| | | | | | | | | | (5.33 ± 0.11) |

>denotes less than 50% inhibition in the reported highest concentration and
>>denotes no inhibition at the reported concentration

TABLE 3
C-3 substituted analogs of compound 1. IC₅₀ (pIC₅₀ ± SEM) are reported.

| Formula | ID | Structure | R | PARP2 | | TNKS2 | | PARP10 | PARP15 |
|---|---|---|---|---|---|---|---|---|---|
| XX | 19 | 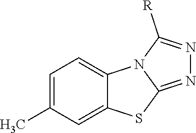 | —OH | >100 | μM | >100 | μM | 5.4 μM (5.27 ± 0.13) | >10 μM |
| XXI | 20 | | —SH | >10 | μM | >100 | μM | 1.5 μM | >10 μM |
| | | | | | | | | (5.81 ± 0.10) | |
| XXV | 21 | | 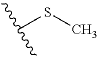 [SMILES: *SC] | 12 | μM | >100 | μM | 3.7 μM (5.45 ± 0.54) | >10 μM |
| III | 22 | | —NH₂ | 1.6 | μM | 5.7 | μM | 0.18 μM* | 0.30 μM |
| | | | | | | | | (6.73 ± 0.12) | (6.53 ± 0.14) |
| XXII | 23 | | 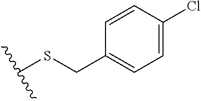 [SMILES: *SCc1ccc(Cl)cc1] | >100 | μM | >100 | μM | >10 μM | >10 μM |
| XXIV | 24 | | 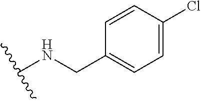 [SMILES: *NCc1ccc(Cl)cc1] | 70 | μM | >100 | μM | >>10 μM | >10 μM |
| XXIII | 25 | | 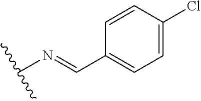 [SMILES: */N=C/c1ccc(Cl)cc1] | 2.9 | μM | >100 | μM | >10 μM | 7.9 μM |
| XXXV | 101 | 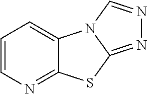 [SMILES: c1cnc2sc3nncn3c2c1] | | ND | | ND | | 30 μM | ND |
| XXVI | 26 | 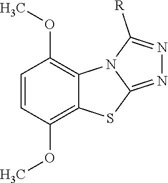 | —SH | 19 | μM | 66 | μM | >>10 μM | >>10 μM |
| XXVII | 27 | | 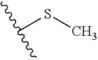 [SMILES: *SC] | 8.4 | μM | >100 | μM | >>10 μM | >>10 μM |
| II | 28 | | —NH₂ | 10 | μM | 10 | μM | 0.12 μM* | 0.12 μM* |
| | | | | | | | | (6.93 ± 0.06) | (6.91 ± 0.03) |
| XXVIII | 29 | | 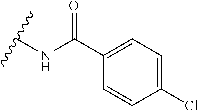 [SMILES: *NC(=O)c1ccc(Cl)cc1] | 44 | μM | 20 | μM | >>10 μM | 7.9 μM |
| XXXIV | 30 | | 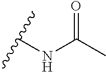 [SMILES: *NC(C)=O] | 53 | μM | >100 | μM | >>10 μM | >>10 μM |
| XXXIII | 31 | | 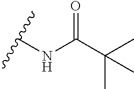 [SMILES: *NC(=O)C(C)(C)C] | 97 | μM | >100 | μM | >>10 μM | >>10 μM |
| XXXI | 102 | 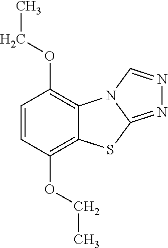 [SMILES: CCOc1ccc(OCC)c2c1sc1nncn12] | | ND | | ND | | 610 nM (6.21 ± 0.05) | ND |
| XXXII | 103 | 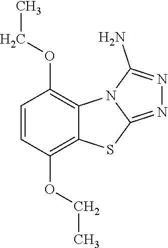 [SMILES: CCOc1ccc(OCC)c2c1sc1nnc(N)n12] | | ND | | ND | | 72 nM (7.14 ± 0.20) | ND |
| XXXVI | 104 | 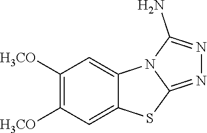 [SMILES: COc1cc2sc3nnc(N)n3c2cc1OC] | | ND | | ND | | 110 nM (6.97 ± 0.13 | 170 nM (6.77 ± 0.04) |
| XXXVII | 105 | 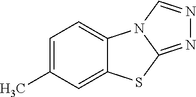 | | ND | | ND | | >>10 μM | >10 μM |
| XXXVIII | 106 | 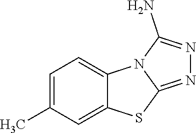 | | ND | | ND | | 60 nM (7.26 ± 0.06) | 1.2 μM |
| XXXIX | 107 | 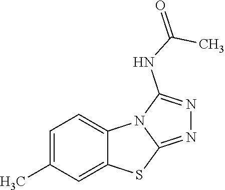 [SMILES: CC(=O)Nc1nnc2sc3cc(C)ccc3n12] | | ND | | ND | | 2.6 μM | 5.2 μM |
| XL | 108 | 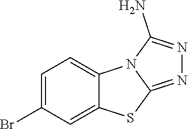 [SMILES: Nc1nnc2sc3cc(Br)ccc3n12] | | ND | | ND | | 60 nM (7.22 ± 0.11) | 150 nM (6.84 ± 0.08) |
| XLI | 109 | 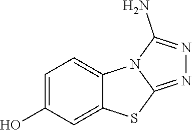 [SMILES: Nc1nnc2sc3cc(O)ccc3n12] | | 5-10 | nM | ~200 | nM | 220 nM (6.66 ± 0.12) | 680 nM (6.17 ± 0.12) |

| | | | | | | | |
|---|---|---|---|---|---|---|---|
| XLII | 110 | 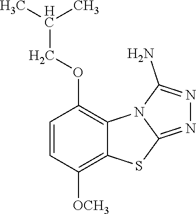 [SMILES: COc1ccc(OCC(C)C)c2c1sc1nnc(N)n12] | 31 | μM | ND | 80 nM (7.09 ± 0.03) | 180 nM (6.74 ± 0.11) |

>denotes less than 50% inhibition in the reported highest concentration and
>>denotes no inhibition at the reported concentration,
*value limited by protein concentration used.
ND = not determined

TABLE 4
Profile of the selected compounds against the PARP
enzymes IC₅₀ (pIC₅₀ ± SEM, n = 3) where mono-ARTs
are measured using a proximity enhanced assay.

| | 10 (OUL245) | 22 (OUL243) | 28 (OUL232) |
|---|---|---|---|
| | (IV) | (III) | (II) |
| PARP1 | 570 nM | 1.6 μM | 15 μM |
| | (6.25 ± 0.02) | (5.79 ± 0.03) | |
| PARP2 | 44 nM | 1.6 μM | 10 μM |
| | (7.44 ± 0.12) | | |
| PARP3 | 8.8 μM | 34 μM | 50 μM |
| PARP4 | 6.0 μM | 10 μM | 11 μM |
| TNKS1 | 1.6 μM | 2.1 μM | 5.4 μM |
| TNKS2 | 370 nM | 5.7 μM | 10 μM |
| | (6.43 ± 0.13) | | |
| PARP7 | >>10 μM | 3.8 μM | 83 nM |
| | | | (7.08 ± 0.06) |
| PARP10 | 2.9 μM | 25 nM | 7.8 nM |
| | | (7.60 ± 0.03) | (8.11 ± 0.12) |
| PARP11 | 9.4 μM | 470 nM | 240 nM |
| | | (6.33 ± 0.13) | (6.61 ± 0.07) |
| PARP12 | >10 μM | 4.4 μM | 160 nM |
| | | | (6.80 ± 0.002) |
| PARP14 | 6.7 μM | 650 nM | 300 nM |
| | | (6.19 ± 0.03) | (6.52 ± 0.03) |
| PARP15 | 2.0 μM | 260 nM | 56 nM |
| | | (6.59 ± 0.11) | (7.25 ± 0.01) |

>denotes less than 50% inhibition in the reported highest concentration and
>>denotes no inhibition at the reported concentration

### CITATION LIST

### Patent Literature

WO2017174879

### Non Patent Literature

Galera-Prat, A., Morais, S., Vazana, Y., Bayer, E. A. and Carrión-Vázquez, M. (2018) Journal of Biological Chemistry, 293, 7139-7147
Maksimainen M M, Murthy S, Sowa S T, Galera-Prat A, Rolina E, Heiskanen J P & Lehtio L (2021) Analogs of TIQ-A as inhibitors of human mono-ADP-ribosylating PARPs. Bioorg Med Chem: 116511
Mehta C C, Bhatt H G. Tankyrase inhibitors as antitumor agents: a patent update (2013-2020). Expert Opin Ther Pat. 2021 July; 31(7):645-661
Narwal M, Fallarero A, Vuorela P & Lehtio L (2012) Homogeneous screening assay for human tankyrase. J Biomol Screen 17: 593-604
Velagapudi U K, Patel B A, Shao X, Pathak S K, Ferraris D V, Talele T T. Recent development in the discovery of PARP inhibitors as anticancer agents: a patent update (2016-2020). Expert Opin Ther Pat. 2021 July; 31(7):609-623
Venkannagari H, Fallarero A, Feijs K L, Lüscher B & Lehtiö L (2013) Activity-based assay for human mono-ADP-ribosyltransferases aimed at screening and profiling inhibitors. Eur J Pharm Sci 49: 148-156
Venkannagari H, Verheugd P, Koivunen J, Haikarainen T, Obaji E, Ashok Y, Narwal M, Pihlajaniemi T, Lüscher B, Lehtiö L (2016) Small-molecule chemical probe rescues cells from mono-ADP-ribosyltransferase ARTD10/PARP10-induced apoptosis and sensitizes cancer cells to DNA damage. Cell Chem Biol 23(10):1251-1260.

1. A method of treating cancer or an inflammatory disorder comprising administering a compound having a general formula: 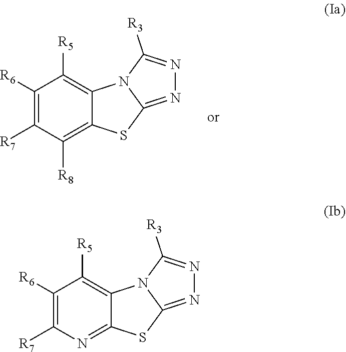
wherein:
R3, R5, R6, R7 and R8 are independently selected from the group consisting of: H, —NH₂, —COOH, —CN, —NO₂, —SH, —OH, F, Cl, Br, I, C₁₋₄ alkyl, aryl, C₁₋₄ alkyl aryl, C₁₋₄ alkyl halide, C₁₋₄ amino alkyl, aryl halide, C₁₋₄ alkyl aryl halide, alkoxy —OR, ester —OC(═O)R, amine —NHR, amide —NHC(═O)R, imine —(N=)R, NHSO₂(C₁₋₄ alkyl), and thioether —SR;
wherein R is selected from the group consisting of: C₁₋₄ alkyl, aryl, C₁₋₄ alkyl aryl, C₁₋₄ alkylhalide, aryl halide, C₁₋₄ amino alkyl, C₃₋₆ cycloalkyl and C₁₋₄ alkyl aryl halide;
or a pharmaceutically acceptable salt thereof
to a patient suffering from a cancer or an inflammatory disorder.

2. The method according to claim 1, wherein
R3 is selected from the group consisting of: H, —NH₂, —SH, —OH, F, Cl, Br, I, C₁₋₄ alkyl, aryl, C₁₋₄ alkyl aryl, C₁₋₄ alkyl halide, aryl halide, C₁₋₄ alkyl aryl halide, C₁₋₄ alkoxy halide, aryloxy halide, alkoxy —OR, ester OC(═O)R, amine —NHR, amide —NHCOR, imine —(N=)R, and thioether —SR;
wherein R is selected from the group consisting of: C₁₋₄ alkyl, aryl, C₁₋₄ alkyl aryl, C₁₋₄ alkylhalide, aryl halide, and C₁₋₄ alkyl aryl halide; and
R5, R6, R7 and R8 are independently selected from the group consisting of: H, F, Cl, Br, I, C₁₋₄ alkyl, C₁₋₄ alkoxy, C₁₋₄ alkyl halide, C₁₋₄ alkoxy halide, and OH;
or a pharmaceutically acceptable salt thereof.

3. The method according to claim 2, wherein
R₃ is selected from the group consisting of: H, NH₂, SH, OH, amine —NHR, amide —NHCOR, imine —(N=)R, and thioether —SR, wherein R is selected from the group consisting of: C₁₋₄ alkyl and C₁₋₄ alkyl aryl halide.

4. The method according to claim 2, wherein:
R₅ is selected from the group consisting of: H, OH, C₁₋₄ alkyl, C₁₋₄ alkoxy and F;
R₆ is selected from the group consisting of: H, C₁₋₄ alkyl and C₁₋₄ alkoxy;
R₇ is selected from the group consisting of: H, OH, C₁₋₄ alkyl, C₁₋₄ alkoxy and Cl; and
R₈ is selected from the group consisting of: H, OH, C₁₋₄ alkyl, and —C₁₋₄ alkoxy.

5. The method according to claim 4, wherein said C₁₋₄ alkyl is methyl, ethyl or iso-propyl.

6. The method according to claim 4, wherein said C₁₋₄ alkoxy is methoxy or ethoxy.

7. The method according to claim 4, wherein at least two of the groups R₅—R₈ are H.

8. The method according to claim 1, wherein the imine is:

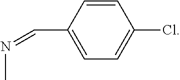

9. The method according to claim 1, wherein the amine is:

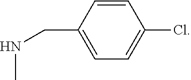

10. The method according to claim 1, wherein the amide is:

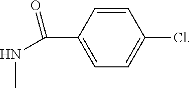

11. The method according to claim 1, wherein the thioethers are selected from the group consisting of:

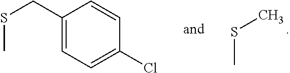

12. The method according to claim 1, wherein:
R₃ is an amino group; and
R₅ and R₈ are methoxy groups.

13. The method according to claim 12, the compound having a formula:

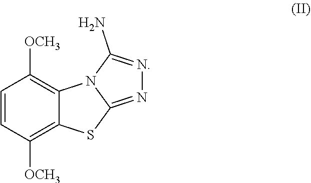 [SMILES: COc1ccc(OC)c2c1sc1nnc(N)n12]

14. The method according to claim 1, wherein
R₃ is an amino group; and
R₇ is a methyl group.

15. The method according to claim 14, the compound having a formula:

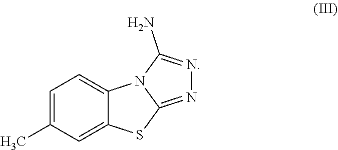

16. The method according to claim 1, wherein
R₇ is a hydroxyl group.

17. The method according to claim 16, the compound having a formula:

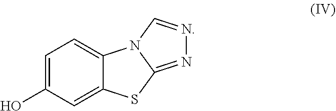 [SMILES: Oc1ccc2c(c1)sc1nncn12]

18. The method according to claim 1, the compound having a formula:

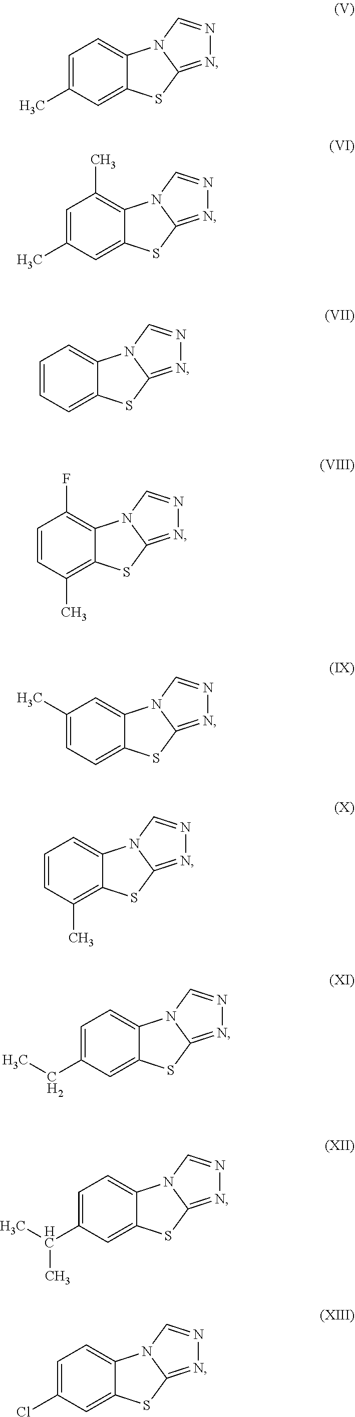

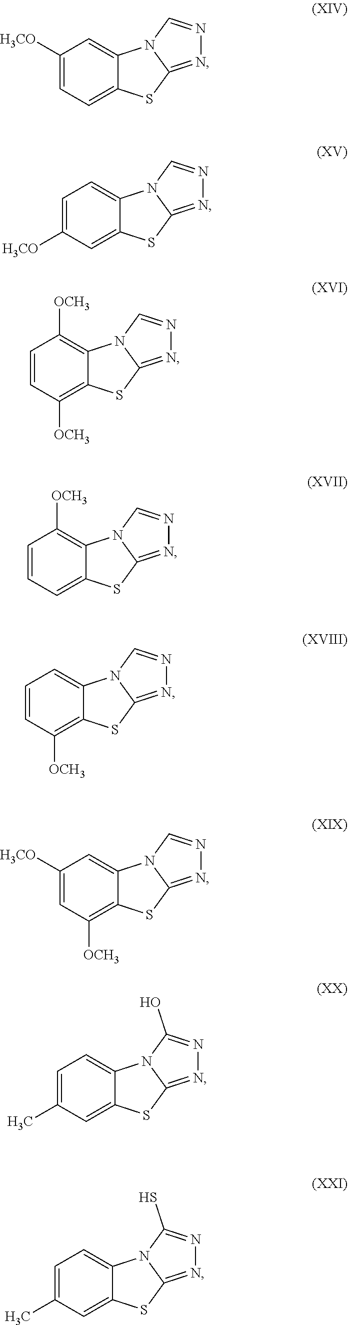

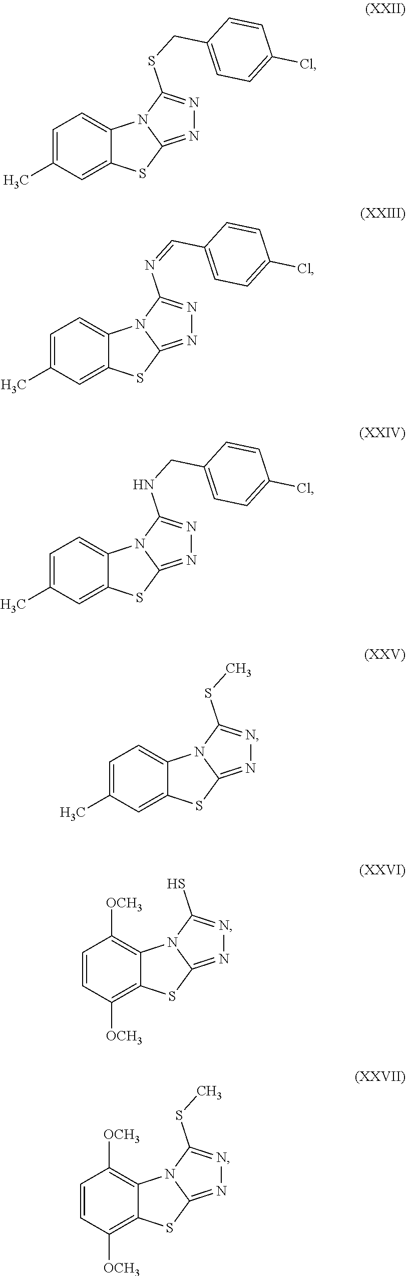 [SMILES: Cc1ccc2c(c1)sc1nnc(SCc3ccc(Cl)cc3)n12 ; Cc1ccc2c(c1)sc1nnc(/N=C\c3ccc(Cl)cc3)n12 ; Cc1ccc2c(c1)sc1nnc(NCc3ccc(Cl)cc3)n12 ; CSc1nnc2sc3cc(C)ccc3n12 ; COc1ccc(OC)c2c1sc1nnc(S)n12 ; COc1ccc(OC)c2c1sc1nnc(SC)n12]

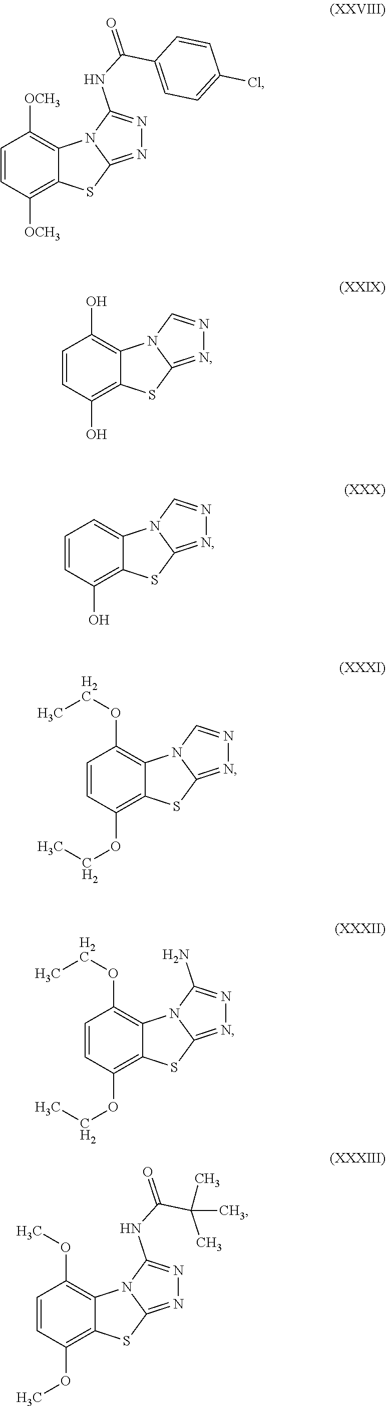 [SMILES: COc1ccc(OC)c2c1sc1nnc(NC(=O)c3ccc(Cl)cc3)n12 ; Oc1ccc(O)c2c1sc1nncn12 ; Oc1cccc2c1sc1nncn12 ; CCOc1ccc(OCC)c2c1sc1nncn12 ; CCOc1ccc(OCC)c2c1sc1nnc(N)n12 ; COc1ccc(OC)c2c1sc1nnc(NC(=O)C(C)(C)C)n12]

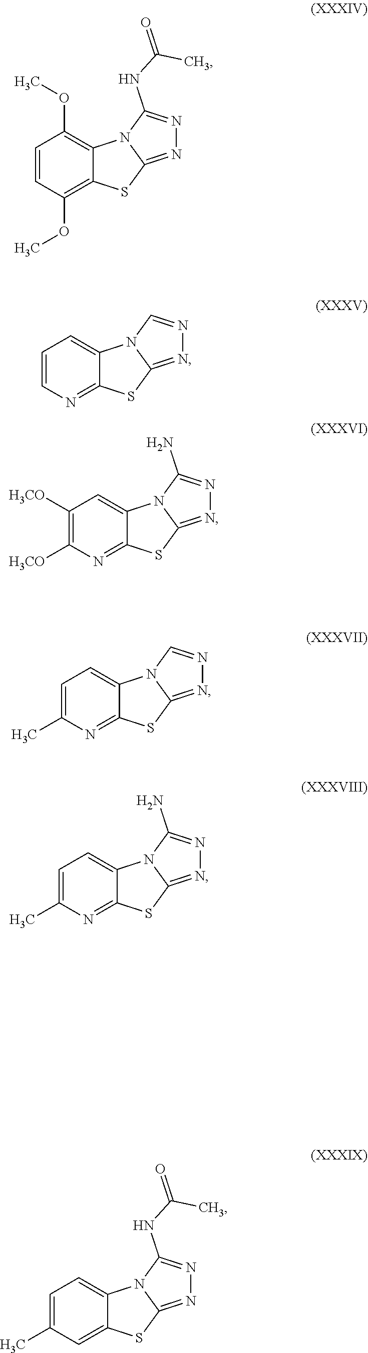 [SMILES: COc1ccc(OC)c2c1sc1nnc(NC(C)=O)n12 ; c1cnc2sc3nncn3c2c1 ; COc1cc2c(nc1OC)sc1nnc(N)n12 ; Cc1ccc2c(n1)sc1nncn12 ; Cc1ccc2c(n1)sc1nnc(N)n12 ; CC(=O)Nc1nnc2sc3cc(C)ccc3n12]

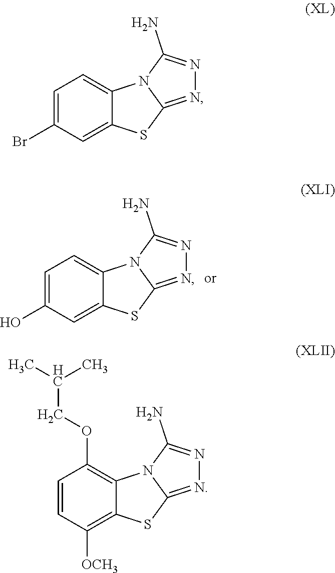

19-23. (canceled)

24. The method according to claim 1, wherein the compound is administered along with at least a pharmaceutically acceptable buffer, carrier, excipient, preservative or stabilizer.

25-38. (canceled)

39. The method according to claim 1,
wherein the following combinations of R3-R8 substitutions are excluded:
R3 is H, R5 is H, R6 is H, R7 is H, and R8 is H;
R3 is H, R5 is H, R6 is H, R7 is —CH₃, and R8 is H;
R3 is H, R5 is H, R6 is H, R7 is Cl, and R8 is H;
R3 is H, R5 is H, R6 is H, R7 is —OH, and R8 is H;
R3 is H, R5 is H, R6 is H, R7 is —OCH₃, and R8 is H;
R3 is H, R5 is —CH₃, R6 is H, R7 is-CH₃, and R8 is H;
R3 is —OH, R5 is H, R6 is H, R7 is —CH₃, and R8 is H; and
R3 is —SH, R5 is H, R6 is H, R7 is —CH₃, and R8 is H.

40-44. (canceled)
